(19) United States
(12) Patent Application Publication
(10) Pub. No.: US 2005/0148085 A1
(43) Pub. Date: Jul. 7, 2005

(54) Method and a system for detecting and optinally isolating a rare event particle

(75) Inventor: Rasmus Dines Larsen, Frederiksberg (DK)

Correspondence Address: GIFFORD, KRASS, GROH, SPRINKLE & CITKOWSKI, P.C, PO BOX 7021, TROY, MI, 48007-7021, US

(73) Assignee: CHEMOMETEC A/S, Allerod (DK)

(21) Appl. No.: 10/489,930

(22) PCT Filed: Sep. 16, 2002
(86) PCT No.: PCT/DK02/00603

(30) Foreign Application Priority Data
Sep. 16, 2001 (DK) PA 2001 01346

(51) Int. Cl. G01N 33/48

(52) U.S. Cl. 436/63; 422/73

(57) ABSTRACT

The invention relates to the field of detecting and optionally collecting and isolating rare event particles. The method according to the invention is based on relatively simple optical equipment, which requires a few and uncomplicated decisions for the user of the system. The method is based on the acquisition of an image of relatively low resolution and magnification of a large volume of sample and detecting the presence or absence of the rare event particle. The method is then repeated with at least one further volume of sample. The method is particularly adapted for detection of white blood cells in leuko-depleted blood.

### TECHNICAL FIELD

This application is a non-provisional claiming priority from Danish patent application No. PA 2001 01346 filed 16 Sep. 2001, which is hereby incorporated by reference in its entirety. All patent and non-patent references cited in that application, or in the present application, are also hereby incorporated by reference in their entirety.

The invention relates to the field of detecting and optionally collecting and isolating rare event particles.

### PRIOR ART

Methods and systems for the detection of rare event particles are known from the prior art. Most of these methods are based on analysis of samples containing rare event particles in flow cytometers or cell analysers. In a flow cytometer, the sample liquid is moved by a carrier stream at high speed through a detection area, where the particles normally are illuminated and electromagnetic radiation from the particles are detected by e.g. a CCD. The sample liquid is preferably so thin that only or substantially only one particle passes the detection area at a time. Furthermore, the sample liquid is moved at very high speed (meters per second). As a consequence of this, the typical acquisition rate of flow cytometers is approx 5.000-10.000 events per second. From this it follows that the time for accumulation of electromagnetic radiation from any one particle during its stay in the detection area is extremely short. This results in a low signal/noise ratio.

EP 0 608 987 A1 (BECTON DICKINSON AND CO.) concerns a method for labelling of cells for detection of rare events, which occur at a frequency of less than one in 10⁶ cells. The method comprises labelling the cells with a first marker specific for the rare event particle and labelling the cells with at least a second marker specific for the majority of the other cells. Rare event particles are detected by analysing the particles for the presence of the first marker and the absence of the second marker. The analyses are carried out in a flow cytometer. This teaching tries to solve the problem of detecting rare particles by increasing the difference in signal between rare and frequent particles. The reference does not address the problem of identifying rare particles in the absence of other particles.

U.S. Pat. No. 4,765,792 (ONCOR INC.) discloses an image analysis method for detecting rare cells in a biological sample. A colour image of the sample is decomposed into its colour components, e.g. red, blue, and green. The locations of rare cells in the image are then found using colour filtering and masks. By finally employing knowledge about the shape and/or size of the rare cells, many artefacts can be filtered away. The samples to be analysed are placed on a microscope slide on a x-y stage. The method is thus a semi-manual method which requires manual preparation of the sample before it is exposed to the image analysis. This method cannot be used when the size and/or shape of the rare cell is not known.

U.S. Pat. No. 6,004,821 (LEVINE & WARDLAW) discloses a container for urine analysis, which comprises a rare event detection chamber. In the rare event detection chamber, essentially all the water from the urine sample is absorbed by a filter or a gel, the rare event particles are stained and they are absorbed on a surface. After automatic scanning the number and type of rare event particles are determined. The method is exclusively adapted for used with urine samples and suffers from the disadvantage that the detected rare event particle cannot be isolated and analysed further, since it is absorbed on a surface.

WO 95/13540 (BECTON DICKINSON AND CO.) discloses a method for counting rare cells. Rare cells are defined as cells making up less than 5% of the total number of cells in a sample. The cells in a sample such as peripheral blood or bone marrow are labelled with one fluorescent marker reacting with all cells in the sample and another fluorescent marker reacting selectively with the rare cells. Finally a known number of fluorescent beads are added to the sample. The sample is analysed using a cell analyser or a cell sorter and the number of rare cells is determined. This method thus requires expensive equipment. Such instruments can only be operated accurately by trained personnel, since a number of choices have to be made by the operator and these choices in turn affect the accuracy of the assessment.

WO 99/57955 (LUMINEX CORP.) discloses a method for data collection in a flow analyser such as a flow cytometer. The method comprises receiving incoming data, storing the data in a circular buffer, directing the reading of data by a processor, receiving the data in the processor and collecting and processing the data with substantially zero dead time. The method is adapted for use when the sampling periods are about one millionth second or less such as in a flow cytometer.

WO 00/49391 (BIO-VIEW LTD.) disclose a method and a system for detection of rare cells, such as rare foetal cells in maternal blood. The rare cells are detected using two algorithms, one which identifies rare cells based on morphology criteria, and another algorithm, which identifies the rare cells based on cellular markers such as chromosomal markers, antigen markers and/or chemical/biochemical markers. Only cells detected by both algorithms are classified as belonging to the group of rare cells. The system was capable of identifying 30 objects among 200,000 maternal white blood cells. Of these one third turned out to be artefacts. This method requires knowledge about the shape and size of the rare event particles as well as knowledge about the presence of cellular markers in the particles.

U.S. Pat. No. 5,037,207 (OHIO STATE UNIVERSITY RESEARCH) concerns a laser imaging system capable of scanning targets of any size. The laser beam may be directed by a beam controller to any one of 16 million locations on a target within an accuracy of +/−0.5 μm. The system is easily adaptable for detection of rare events. According to the disclosure, it is capable of detecting a single positive fluorescent cell on a slide area of 400 sq. mm, which can include 20 million cells. The system thus appears to be adapted for used with cells that are immobilised or essentially immobilised on a microscope slide.

Thus it is an object of the present invention to provide a simple and automated method or detection of rare event particles in a liquid sample, in which it is possible to increase the signal to noise ratio compared to the methods used in flow cytometry. It is a further object to provide a method which does not require extensive training of the operator.

### DEFINITIONS

Sample: a representative portion of the total volume of liquid sample to be analysed.

Exposure: Exposures according to the present invention is carried out by detecting the intensity of electromagnetic radiation by individual detection elements, such as by a charge coupled device. By one exposure is meant one period of accumulation of electromagnetic radiation by the detection elements. One exposure may comprise several frame grabbing actions. The grabbed images may be averaged to produce one averaged image, which may then be analysed.

Spatial image representation: information being spatially resolved in one or two dimensions. The information results from the detection of electromagnetic radiation, which may be presented in the form of an image.

### SUMMARY OF THE INVENTION

i) According to a first aspect, the invention relates to a method for detecting a rare event particle in a liquid sample comprising the steps ofin a sample device arranging a precisely defined volume of at least 0.1 μl of a liquid sample in an exposing domain of a sample compartment, allowing electromagnetic radiation from the rare event particle(s) in the exposing domain to pass to the exterior,
ii) arranging the sample device in relation to a detection device so that signals from the exposing domain can pass to an array of detection elements in the detection device,
iii) detecting electromagnetic signals from the first volume of liquid sample in the exposing domain by forming a spatial image of the rare event particle(s) on the array of detection elements,
iv) repeating steps i) and iii) at least once for new volumes of the same liquid sample,
v) correlating the spatial image to the number of rare event particle(s) in the volume of liquid sample in the exposing domain.

The present invention relates to detection of rare particles, which occur so rarely that when analysing the sample as defined in claim 1, there are instances where there are not particles present in the volume present in the exposing domain. Expressed as a probability of the occurrence of at least one exposure without any particles this probability is at least 2%. More preferably the probability of no particles in one exposure is at least 3%, more preferably at least 10%, more preferably at least 15%, such as at least 20%, for example at least 25%, such as at least 40%, for example at least 50%, such as at least 60%, for example at least 75%, such as at least 80%, for example at least 90%, such as at least 95%, for example at least 99%, such as 100%.

Expressed in another way, a rare event particle may be defined as a particle occurring less frequently than 10,000 particles per ml of sample liquid, more preferably less than 1,000 particles per ml of sample liquid, more preferably less than 100 particles per ml, for example less than 10 particles per millilitre of sample liquid, such as less frequently than 4 particles per millilitre.

With a volume of 0.1 μl of sample in the exposing domain and no dilution, 10,000 particles per ml corresponds to 1 particle per exposure. The probability of no particles in one exposure is thus substantial, equivalent to about 36%.

Under these conditions, any image of the exposing domain is likely to contain 0 or just 1, 2, 3 or a few objects, which can be identified as particles.

By serial or parallel analysis of a number of volumes of sample each comprising at least 0.1 μl, a substantially large volume of liquid sample may be analysed in a very simple manner. In contrast to flow cytometry, the steps of arranging the sample and detecting the signals is separated in two steps. Thereby the time used for detection of signal from a particle in the sample liquid can be increased and the signal to noise ratio be increased substantially.

In contrast to techniques based on image analysis of samples on a microscope slide, it is possible to perform the sample handling steps of the present analysis completely or partly automatically. The sample may thus be loaded into the sample device through automatic or manual operation of the device, and after being arranged in relation to the detection device, the remaining steps of detection and reloading of sample into the exposing domain may be performed fully automatically.

The steps i) and iii) may be repeated a predetermined number of times, such as a number of times until a predetermined volume of sample has been analysed. This embodiment is especially useful when the method is used for ascertaining that the concentration of the rare event particle is below a certain threshold such as in the analysis of depleted blood, which should not contain more than a certain amount of white blood cells per ml of blood.

According to another embodiment of the invention the steps i) and iii) are repeated a number of times until a predetermined statistical requirement is fulfilled. Such a pre-determined statistical requirement could e.g. be a probability that the particle is absent or present in or below a certain concentration.

For all aspects of the present invention, the purity of the system used for the assessment is very important, since any impurities present may contribute to false positives such as dust particles, which may be identified in the picture as rare event particles. In systems using a stationary flow system it is also of great importance to assure conditions, under which any particle or impurity originally contained in a previous sample is not present or detected in the analysis of subsequent samples.

According to another aspect the invention relates to a method for isolation of a rare event particle comprising
i) arranging a volume of a liquid sample in the exposing domain of a sample compartment,
ii) detection the absence or presence of a rare event particle,
iii) in case of presence of a rare event particle, flowing the volume of sample to an outlet using a carrier liquid, obtaining a sample comprising a rare event particle,
iv) diluting the sample containing collected rare event particles and arranging a volume of the diluted sample comprising the rare event particle in the exposing domain of a sample compartment,
v) repeating steps ii) to iv) until the rare event particle is essentially the only particle in a volume, obtaining a sample comprising essentially only rare event particle(s).

According to this aspect of the invention, it is possible to isolate a rare event particle from other non-rare particles present in the sample using serial dilution of the sub-volume containing the rare event particle.

The repetition(s) of steps ii) to iv) may be carried out in the sample compartment of i) (serial operation) or in a different but often identical sample compartment (parallel operation).

According to a further aspect the invention relates to a method for collection of a rare event particle comprising
i) arranging a volume of a liquid sample in the exposing domain of a sample compartment,
ii) detecting the absence or presence of a rare event particle,
iii) in case of presence of at least one rare event particle, flowing the volume of ample to an outlet, obtaining a sample comprising at least one rare event particle,
iv) repeating steps ii) to iii) until at least a predetermined number of rare event particles is obtained or until a predetermined volume of liquid sample has been analysed in the exposing domain.

The repetition(s) of steps ii) to iii) may as above be carried out in serial or parallel operation.

According to this aspect of the invention it is possible to obtain a sub-sample comprising the rare event particle(s) present in the sample.

According to a further aspect, the invention relates to a system for isolation of a rare event particle comprising
i) a sample compartment comprising an exposing domain, from which electromagnetic radiation from a precisely defined volume of sample can pass to the exterior,
ii) a flow system comprising an inlet and an outlet, at least one of which comprises a stop valve,
iii) pumping means to pump liquid sample or carrier liquid into and through the sample compartment,
iv) the flow system further comprising on the inlet side, at least a sample tube and a carrier liquid tube and valve means to connect the inlet to either of the tubes,
v) the flow system further comprising on the outlet side at least a waste tube and a rare event particle tube, as well as valve means to direct the sample to either of these tubes.

The system is adapted for use in the method for isolation of a rare event particle.

According to a further aspect, the invention relates to a system for collection of rare event particles comprising
i) a sample compartment comprising an exposing domain, from which electromagnetic radiation from a precisely defined volume of sample can pass to the exterior,
ii) a flow system comprising an inlet and an outlet, at least one of which comprises a stop valve,
iii) pumping means to pump liquid sample into and through the sample compartment,
iv) the flow system further comprising on the outlet side at least a waste outlet and a rare event particle outlet, as well as valve means to direct the sample to either of these outlets.

### DRAWINGS

FIG. 1 shows a one sided excitation system.

FIG. 2 shows a cross-section of the excitation light filter in a plane parallel to the sample plane.

FIG. 3 shows the collection angle C and the angle E between the excitation main light path and the detection-sample axis.

FIG. 4 shows a double-sided excitation/detection system.

FIG. 5 shows a double-sided excitation system.

FIG. 6 shows a double-sided detection system.

FIG. 7 shows a schematic illustration of a system adapted to isolate a rare event particle.

FIG. 8 shows a schematic illustration of a system adapted to identify rare event particle.

FIG. 9 shows a schematic illustration of a system adapted to sample preparation and identification of rare event particle.

FIG. 10 shows a schematic illustration of a sample compartment.

FIG. 11 shows the result of the comparison of method according to the present invention and a commercially available instrument used for the assessment of rare events I leucodepleted blood or blood product.

FIG. 12 shows a graph illustrating the observed and/or reported CV for various methods used for the assessment of rare events.

FIG. 13 shows a graph illustrating improved sensitivity.

FIG. 14 shows a suitable optical system for performing detection of rare event particles within a detection unit.

### DETAILED DESCRIPTION OF THE INVENTION

The method and system described in the present application has several uses, which all relate to the detection of rare event particles. In this context a rare event particle may either be a particle which is present in very low concentrations (such as less frequently than 10,000 particles per ml of sample liquid, more preferably less than 1,000 particles per ml of sample liquid, more preferably less than 100 particles per ml, for example less than 10 particles per millilitre of sample liquid, such as less frequently than 4 particles per millilitre) or whose frequency is low in comparison to the prevailing particles in the sample, such as being present in a frequency below 1%, such as below 1 0/00, for example below 1 in 10⁴, such as below 1 in 10⁵, for example below 1 in 10⁶, such as below 1 in 10⁷, for example below 1 in 10⁸.

Of the several possible applications, the invention particularly relates to but is not limited to the following:
detection of cancer cells, or micro-metastases
detection of rare or abnormal cells in a biological sample (which could also be a cancer cell or a micro-metastase in blood or lymph liquid)
detection of foetal cells in maternal blood
identification of infectious diseases such as vira or fungi, which are difficult or impossible to culture
analysis of blood samples such as leukocyte depleted blood, donor blood, a biopsy, maternal blood, blood products, or foetal blood.
quality control in the manufacture of leukocyte depleted blood, which is donor blood depleted of white blood cells.
in urine analysis: detection of proteinaceous casts as indicators of bleeding in the kidneys, or infection in the urine bladder.
detection of other types of particles in urine: red and white blood cells, bacteria, crystals, fecal matter, parasites, spermatozoa, cancer cells, or micro-metastates, ova from parasites.
analysis of drinking water: absence of bacteria,
analysis of waste water: absence of pathogenic bacteria,
analysis of petrol, and oil

From the above it follows that the rare event particles may comprise abnormal cells, cancer cells, micrometastasis, parasites, ova from parasites, blood cells, leucocytes, erythrocytes, blood plates, virus, fungus, fetal cells, foetal blood cells, proteinaceous casts, plasmodium.

The average particle diameter may be less than 20 μm, for example less than 15 μm, such as less than 10 μm, for example less than 5 μm, such as less than 3 μm, for example less than 2 μm, such as less than 1 μm, for example less than 0.5 μm, such as less than 0.2 μm, for example less than 0.1 μm.

Stains

In connection with many embodiments of the method of the invention, the rare event particles, which are to be determined are not in themselves capable of emitting or interacting with an electromagnetic irradiation in a way which can be used as a basis for the image generation and it is therefore often necessary to add one or more components, in the following called reaction components, to the liquid sample prior to the detection. Preferably the addition of one or more reaction components to the sample is performed in the sample device, although the sample may be stained before it is loaded into the sample device. It is often preferred that the signal which is emitted from particles in the device is a photoluminescence signal, originating from a molecule, or a fraction of a molecule having fluorophor properties, naturally contained within or on the particle which is measured.

Signal from Added Chemicals

The signal which is emitted from or transmitted through the sample device often originates from, or is modified by, one or several types of molecules of types which bind to, are retained within, or interact with, rare event particles, such molecules being added to the sample before or during exposure, the molecules being molecules giving rise to one or several of the following phenomena: attenuation of electromagnetic radiation, photoluminescence when illuminated with electromagnetic radiation, scatter of electromagnetic radiation, raman scatter. In the presently most preferred embodiments an effective amount of one or more nucleic acid dyes and/or one or more potentiometric membrane dyes is added.

For example, a particularly important example is a fluorochrome which can be bound to, or retained within, relevant rare event particles so that the particles, upon excitation with a suitable source of electromagnetic irradiation, will emit an electromagnetic irradiation on the basis of which the image can be generated. Such reaction components can suitably initially be loaded in a compartment or flow channel part of the flow system of the sample device from where they can be added to at least a portion of the volume of the liquid sample.

The reaction components, which normally comprise one or more chemicals, are preferably initially loaded in the compartment or flow channel part in solid form. As solid forms of the reaction components may not be easy to dissolve as fast and as efficiently as is necessary for a realistic operation of devices according to the invention, it is often preferred that the reaction components comprise one or more chemicals in solid form in combination with one or more solubilising agents aiding the solubilisation of the chemicals in the liquid sample. The addition of one or more components could have the effect of either control the form the other reaction component have and/or directly taking place in the dissolution or dissolution of the reaction components. Such components having effect of increasing the rate of dissolution or solubilisation of any chemical on a substantially solid, and/or substantially non-aqueous, and/or substantially freeze dried form, are preferably one or more types of organic or inorganic salts.

A very suitable solid form of the reaction components is the freeze-dried form which, because of its high surface area and optionally incorporated solubility enhancing substances show a very high rate of solubility.

The amounts and availability (solubility and/or dispersibility in the liquid sample under the conditions prevailing) of the reaction components and the design of the flow system are preferably so adapted that a predetermined minimum of the reaction components will be contained in the sample present in the sample compartment.

The number of different types of molecules (reaction components) added depends on the complexity of the assessment, and on the nature of the rare event particles being analysed. It is for instance often advantageous to use two or more, such as three or even four types of molecules when the assessment concerns the identification of and differentiation between two or more types of particles (such as a rare event particle and the more frequent particles), where the different particles interact differently with the different molecules, for instance by giving rise to a fluorescent signal at different wavelength. Often the addition of such two or more types of molecules is done simultaneously, but under some conditions it is preferred to add the molecules at different times. These added molecules can interact with the rare event particles for instance by being retained within them, interacting with them or being prepelled by them or in any way alter the properties of the particles or the sample.

Addition of Chemicals

The preferred amount of any chemical component contained in the device prior to analysis can be varied according to the properties of the rare event particles being assessed. The amount can be more than 30 μg per ml of sample, but often it is preferable to have amount of less than 30 μg per ml of sample, even less than 10 μg per ml of sample. Some aspects of this invention allow an amount of less than 1 μg, or even less than 0.1 μg per ml of sample.

Reaction components suited for this purpose are for instance one or more nucleic acid dyes and/or one or more potentiometric membrane dyes. As example of preferred reaction components which can be used to form signals which allow assessment of rare event particles are one or more nucleic acid dyes which is/are selected from the group consisting of: phenanthridines (e.g. ethidium bromide CAS#: 123945-8, propidium iodide CAS#: 25535-164), acridine dyes (e.g. acridine orange CAS#: 65-61-2/CAS#: 10127-02-3), cyanine dyes (e.g. TOTO™-1 iodide CAS#: 143 413-84-7-Molecular Probes, YO-PRO™-1 iodide CAS#: 152 068-09-2-Molecular Probes), indoles and imidazoles (e.g. Hoechst 33258 CAS#: 023 491-45-4, Hoechst 33342 CAS#: 023 491-52-3, DAPI CAS#: 28718-90-3, DIPI (4′,6-(diimidazolin-2-yl)-2-phenylindole)).

In particular it is found that the nucleic acid dye propidium iodide (CAS#: 25535-16-4) is suited for many assessments of DNA containing particles due to the fluorochrome properties which the molecule shows. When the reaction component is a potentiometric membrane dye it can be one or several of the following, but not limited in: Rhodamine-123, Oxanol V. When performing a quantitative assessment of particles it is normally necessary to control the addition of any component to the sample, in order not to affect the result of the assessment, for instance due to variation in dilution. The present invention offers embodiments where such requirements are less important than under conventional situations. This can be accomplished by introducing the components on a form which has only limited effect on the assessment, such as introducing any component as solid matter, thereby substantially not altering the volume of any sample being analysed.

To further enhance the property of any reaction component used to form signals which are detected, or to assure more reliable interaction between the reaction components and the sample or the particles in the sample it can be advantageous to add reaction components, which often are not the direct source of the signals formed hut rather have influence on the signals being formed. One such reaction component, well suited for the assessment of blood cells or bacteria is Triton X-100 (t-Octylphenoxypolyethoxyethanol). The efficiency of such reagent component is often determined by the amount of such reagent component present. In the present invention it is often preferred that amount of interest are between 0.1 and 2% (w/w), preferably between 0.5 and 2%, more preferably between 1 and 1.5%.

In many embodiments other reagent components can be used to stabilise the rare particle, either physically or chemically. Such stabilisation generally has the effect of reducing spatial gradients of any particle within entire volume of the sample, especially during flow through flow channels or tubing, or to reduce the number of “lost” particles, where particles are lost through adhesion to any surface or through disintegration or the like. Many reagent components are useful for this purpose, but in many embodiments it is preferred to use polymer surfactants, such as Pluronic, or citric acid, or salt of citric acid. The preferred quantity of such reagent component is dependent on the nature of the sample and particle being analysed, but often it is preferred that it is between 0.5 and 2% (w/w), such as between 0.5 and 2%, or between 1.0 and 1.5%.

One often preferred embodiment includes one or more particle retaining means capable of selectively and/or substantially reproducibly retaining particles from a volume passed through the particle retaining means. This allows the analysis of large volume of sample. Often the rare particles are detected after it has been released from the particle retaining means, but in other embodiments the particle is detected while still being retained by the particle retaining means.

Depending on the property of the flow system of a sample device and/or the reaction components used, there are virtually unlimited number of different reactions which can take place within a sample device. It would for instance be of great interest to carry out one or more antibody/antigen reactions, preferably also involving polymer beads such as paramagnetic beads, and thus for instance obtain improved accuracy in the identification of one or more types of biological particles such as cells, bacteria and proteins.

Selective Labelling

According to one especially preferred embodiment of the invention labelling of the particles comprise selective labelling of the rare event particle(s) before arranging it in the sample compartment. Thereby it is possible to distinguish the rare event particle from other non-rare particles in the sample. The selective labelling may comprise staining of the rare event particles, such as staining of the nucleus of the rare event particles, preferably a fluorescent staining of the nucleus.

To further improve the possibilities for distinguishing between rare and non-rare particles the method advantageously further comprises selective staining of particles in the sample being non-rare.

The labelling of both the rare and the non rare particles may comprise an antibody based labelling or a stain with a molecular marker linked to a stain.

Example of rare and non-rare particles that may be selectively labelled include but is not limited to the following examples: the non-rare particles may comprise maternal blood cells and the rare particles comprise foetal blood cells, or the non-rare particles may comprise normal mammal tissue cells and the non-rare cells may comprise cancer cells or micrometastases, or the non-rare particles may comprise blood cells and the rare particles may comprise bacteria, fungal cells or spores or virus or plasmodium.

Morphology Criteria

The identification of the rare-event particles may also be performed based on at least one morphological criterion, which identification may advantageously be performed in an automatic image analyser capable of identifying and distinguishing features related to objects in an image.

In most cases, the dimensions of the rare event particles are known. By combining these dimensions with the optical specification of the system (magnification, size of pixels in the array of detection elements), the number of pixels onto which a rare event particles is exposed is known. This knowledge can be used to remove at least part of the noise originating from artefacts such as dust, because such particles often have other dimensions than the rare event particles.

A further parameter which may be used to distinguish rare event particles from noise is integration of signal accumulated by the pixels onto which one particle is exposed. After calibration with known particles this value may also be used to filter away signal from false positives.

Knowledge about both the size and the integrated signal from a particle, can be combined in a treatment of an image to filter away signal from false positives and increase the sensitivity of the method.

Morphological criteria may also be used to distinguish non-rare particles from rare event particles through the use of at least one distinguishing morphological criterion. Finally, the identification of a rare event particle may be performed by combining selective labelling and at least one morphology criterion to identify or distinguish rare event particles from non-rare particles.

Reagent Compartment—Mixing Compartment

In a preferred embodiment of the invention the sample device contains at least one compartment containing chemicals which allows the mixing of the sample material with a solid or liquid material.

The device may comprise several reagent compartments to be used in series for the assessment of several samples or sequential addition to one sample. The several compartments can also be used in parallel for the substantially simultaneous assessment of several samples.

In order to assure fast assessment of a sample it is of interest to be able to perform analysis shortly after the mixing of any chemical components with sample. This time should therefore be less than 60 seconds, or preferably less than 30 seconds or even as low as 15 seconds and in other preferred situations as low as 10 seconds, and preferably as short as 2 seconds or less and even shorter than 1 second.

Gradients

Often it is found especially in relation to mixing a reaction component and a sample material, that gradients of chemical or physical property are formed. Such gradients can be observed in the longitudinal direction of a flow system, defined as parallel to the main direction of flow and/or in a radial direction, defined as perpendicular to the main direction of flow.

Even though it is often of interest to preserve longitudinal gradients in the flow system, it is preferred that such gradients can be eliminated or at least substantially reduced, for instance by passing the liquid sample through a part of a flow channel of the flow system of the sample device having a shape and/or size resulting in substantial reduction of longitudinal gradients in liquids passing therethrough. In the present invention this can be accomplished when at least a part of the flow channel is a flow channel providing substantially laminar flow therethrough and/or comprises one or more mixing chambers.

Similarly, any radial gradient present in the liquid sample in the flow system can be substantially reduced by passing the liquid sample through a part of a flow channel of the flow system of the sample device having a shape and/or size resulting in substantial reduction of radial gradients in liquids passing therethrough. In the present invention this can be accomplished when at least a part of the flow channel has at least one bend or obstruction resulting in substantially turbulent flow in the liquid passing the bend or obstruction.

In order to flow the sample into or within or out of the sample device, there may be at least one propelling means provided in the sample device or in a device with which the sample device can be engaged. In the latter embodiment it is to be understood that the liquid sample is introduced into the device after engagement with the detection means.

In particular it may be of interest that the propelling means is provided in an adapter device with which the sample device is engaged during liquid sample acquisition or even more preferred that the propelling means constitutes an integrated part of the sample device.

Flow Regulation

It is preferred that the velocity of the flow into, within, or out of the sample device is regulated by means of one or more regulating means constituting part of the flow system. Such flow regulating means could be one or more of stop valves, one way valves, and pressure and/or speed reduction valves.

Preferably the flow regulation means is arranged to function stepwise so that the sample and/or the reagent component may be flowed stepwise through the sample device. It is furthermore preferred that at least the step of flowing the sample into the exposing domain is carried out in connection with the engagement of the sample device into the system.

The sample in the sample device can be flown by means of a flow system, which can be driven by a pump or a pressurised gas, preferably air, or by causing a pressure difference such that the pressure on the exterior of the inlet is higher than the pressure within at least a part of the device thus forcing the sample to flow through the inlet. In many embodiments of the present invention the flow in said flow system is controlled by one or more valves which can adjust the flow speed of the sample.

In many preferred situations the flow of liquid in the sample device can be brought about by a vacuum, the vacuum being applied from a reservoir, preferably contained within the device. The vacuum can be established by a mechanical or physical action creating the vacuum substantially simultaneously with the introduction or the movement of the sample. These mechanical or physical actions can be: a peristaltic pump, a piston pump, a membrane pump, a centrifugal pump and a hypodermic syringe.

In most cases, flow in predominately one direction is preferred. It is then of particular interest to use valves which substantially only allow the flow in one direction. Such valves can for instance be placed up- and/or downstream from the sample compartment thus allowing control of the flow condition in the sample compartment.

The outlet from the sample compartment can be passed through a flow controlling means, such as a valve, which only allows gas to pass through. One such type of valves which often is preferred, is one which allows gas and air to pass but can close irreversibly when the valve comes in contact with liquid sample. The effect of such valve is to minimise the movement of any sample within the sample compartment during analysis, thereby obtaining substantial stand still during exposure.

Reproducibility

In order to allow reproducible and reliable assessment of particles it is preferred that the design and the production of the sample device is such that any dimensions of the sample device which influence the volume of sample represented in the spatial image representation are kept within predetermined variations from device to device. On the other hand some aspects of the design and the production of the sample device can be such that variations between individual sample devices in dimensions which influence the volume of sample represented in the spatial image representation are indicated on the sample devices in that each sample device is associated with information as to data concerning the dimensions in question, and the information is taken into consideration in, the processing of the detected image representation. In particular it is preferred that such information as to data concerning the dimensions in question is contained in insignia carried by the sample devices and readable by the detection device or another device adapted to read the insignia.

It is preferred that the transfer of data to the processing means is performed automatically or through human interaction. If the transfer of data to the processing means is performed automatically it is often only performed when an authentication insignia has been identified. Normally such authentication insignia is an image or other insignia proprietary to a producer or distributor of the sample devices authorised by a private or official body to provide the sample devices for the determination or assessment in question. Furthermore, the authentication insignia can comprise encrypted information or a trademark, and the detection device or other device is capable of decrypting the encrypted information or identifying the trademark.

In some embodiments of the invention, variations in dimensions of the sample device which influence the volume of sample represented in the spatial image representation are compensated in the assessment on the basis of volume calibration means. Often such volume calibration means is constituted by one or more of the reaction components or calibration beads, in which case the reaction component or components in question is/are loaded in a predetermined concentration, and the flow operation of the device is performed in a manner ensuring that the predetermined concentration will be reflected in the concentration of the reaction component or components in the exposing domain.

The detection of the spatial image representation of the exposing domain of the sample device is preferably performed by means of an array of active detection elements onto which array the spatial image representation is exposed. In order to facilitate the assessment of particles the intensities detected by the array of detection elements are processed in such a manner that representations of electromagnetic signals from the particles are identified as distinct from representations of electromagnetic background signals.

In many advanced embodiments of the present Invention it is possible to determine the amount and/or the level of any constituent in a sample material, preferably substantially simultaneously with the assessment of rare event particles, and the constituent being determined could be, e.g., one or several of: fat, protein such as haemoglobin, lactose, citric acid, glucose, ketones, carbon dioxide, oxygen, pH, potassium, calcium, sodium. The determination of a component can be done in a sample compartment or a domain, often the same sample compartment or exposing domain which is used for the assessment of rare event particles. The methods used for the determination could be based on spectrophotometric measurement, the spectrophotometric measurement being, e.g., one or several of; mid-infrared attenuation, near-infrared attenuation, visible attenuation, ultra-violet attenuation, photoluminescence, raman scatter, nuclear magnetic resonance. Other methods also suited for the determination of any chemical property could based on potentiometric measurement, preferably by the use of ion selective electrode.

Sample Volume

It is often of interest to minimise the use of any sample material and any chemical component used for the analysis, for instance when the sample material or any chemical reagent can be considered hazardous or when it is difficult to obtain in large quantity. This can be accomplished by the use of the present invention. The optimal volume of the sample needed is highly dependent on the number of particles present in the sample and the predetermined statistical quality parameter sought. Sample volumes as small as 5 ml or less and even as small as 0.02 ml can be used. The volume of the sample needed is highly dependent on the number of particles present in the sample and the predetermined statistical quality parameter sought, whereby typical volumes applied is less than 5 ml of a liquid sample, preferably by using less than 2 ml of a liquid sample, more preferably by using less than 1 ml of a liquid sample, more preferably by using less than 0.5 ml of a liquid sample, more preferably by using less than 0.2 ml of a liquid sample, more preferably by using less than 0.1 ml of a liquid sample, the volume being defined as the total volume of any liquid sample introduced to the sample compartment, or any flow system connected to the sample compartment before or after or during the measurement of the sample.

Other preferred embodiments of the present invention make it possible to assess particles from a considerably large volumes of sample. This can allow the measurement of samples with only few particles of interest per volume of sample (such as in depleted blood). Sample volumes larger than 1 ml can be used for the analysis, the volume being defined as the total volume of any sample introduced to any flow system connected to the sample device before the measurement of the sample.

In many assessments of particles it is of interest to allow exposure of signals from large volumes of sample. The volume of the liquid sample from which signals such as electromagnetic radiation is exposed at one time onto the detection system is normally in the range between 0.1 μl and 100 μl, preferably from 0.5 to 50 μl, such as from 0.5 to 20 μl, more preferably from 0.5 to 5 μl, for example from 0.5 to 4 μl, such as from 0.5 to 1.0 μl, from 1-2 μl, from 3-4 μl, or from 4-5 μl. These volumes are suitable when cells are the rare event particles.

The precisely defined volume may also be from 0.1 to 5 μl, for example from 0.1 to 2.5 μl, such as from 0.1 to 1 μl. These volumes are suitable when bacteria constitute the rare event particles.

Generally the volume of the sample being analysed should be as large as possible. This allows the simultaneous assessment of a large volume of sample, but the optimal volume is often defined by one or more aspects of the detection system and the sample being analysed. Thus the volume of the sample in the sample compartment can be less than 0.1 μl but often volume of more then 0.1 μl, 1.0 μl or even 10 μl is used. In still other application volume of the sample compartment as large as 100 μl or more can be used.

One particular feature of the present invention, is the relatively low magnification used for detection. The advantage of using a low magnification, is that more signal can be recorded from one particle. Furthermore, the focus depth is increased when using low magnification thus allowing detection of particles in a thicker layer. Finally, with a relatively low magnification, a larger volume can be examined in one exposure.

Although the method is not restricted to low degrees of magnification, it is certainly advantageous to use magnification in the order of 1:1. Preferably the ratio of a linear dimension of the image on the array of detection elements to the original linear dimension in the exposing domain is in the range from 10:1 to 1:10. More preferably the ratio of a linear dimension of the image on the array of detection elements to the original linear dimension in the exposing domain in the range from 1.5:1 to 1:2.

Other ratios may likewise be used, so that the ratio of the image of a linear dimension on the array of detection elements to the original linear dimension in the exposing domain may be smaller than 100:1, such as smaller than 40:1, for example smaller than 10:1, such as smaller than 5:1, preferably smaller than 2:1, more preferably smaller than 1:1.

Reduction may also be used such as a reduction given as the ratio of a linear dimension of the image on the array of detection elements to the original linear dimension in the exposing domain. This reduction may be at least 1:2, such as at least 1:3, for example at least 1:4, such as at least 1:5, for example at least 1:10.

Resolution

According to an especially preferred embodiment of the invention, a relatively low resolution is used. This implies that the number of detection elements onto which a single rare event particle in the exposing domain is imaged is relatively low. Under such conditions, details about the shape of the particle normally cannot be determined. The advantage of using low resolution especially when combined with low magnification is that a large volume can be “viewed” by the array of detection elements and more signal can be accumulated from the particles.

More specifically the number of neighbouring detection elements, onto which the image of one rare event particle is exposed is in the range from 1 to 16. More preferably a single rare event particle is exposed onto 3 to 6 neighbouring detection elements.

Dimensions of the Sample Compartment

As mentioned above, it is one of the characterising features of the present invention that a relatively large volume of sample can be exposed to the detection system. The sample is contained in the interior of the sample compartment, which normally has an average thickness of between 20 μm and 2000 μm, usually between 20 μm and 1000 μm and in many practical embodiments between 20 μm and 200 μm.

Normally, the sample compartment has dimensions, in a direction substantially parallel to a wall of an exposing window, in the range between 1 mm by 1 mm and 10 mm by 10 mm, but it will be understood that depending on the design, it may also be larger and, in some cases, smaller.

Thus the area of the exposing window can be as little as 0.1 mm² or more, more preferably with an area of 1 mm² or more, preferably with an area of 2 mm² or more, preferably with an area of 4 mm² or more, preferably with an area of 10 mm² or more, preferably with an area of 20 mm² or more, preferably with an area of 40 mm² or more, more preferably with an area of 100 mm² or more, preferably with an area of 200 mm² or more, preferably with an area of 400 mm² or more, preferably with an area of 1000 mm² or more. Similarly, it is advantageous to extend the window of the sample compartment in a direction which is parallel to the plane of any window exposing signals from the sample to the exterior in order to extend the area of the exposing window and thus increase the volume of the sample which is exposed to the exterior.

The requirements of the wall of the sample compartment are in particular that the wall allows the signals to pass without any significant limitations. In practice no upper limit is given for the wall thickness apart from what is defined by cost and design. The wall is preferably a substantially stable wall, which leads to a lower thickness limit for each material used. Preferably, the wall is from 0.1 mm to 2 mm, such as from 0.5 mm to 1.5 mm, more preferred from 0.75 mm to 1.25 mm.

Exposing Domain

Concerning the spatial definition of the shape and size of the area of an exposing domain or a window exposing signals to the detection device there are at least two feasible methods for substantially reliable definition of the size and shape of this area. The first, and in many embodiments preferred method, is to adapt the detection device to be sensitive to exposed signals from a well defined area of the exposing window, e.g. by adapting any focusing means of the detection device. The second method, which is in particular preferred when it is difficult to adapt the sensing area of the detection device, is to define the boundaries of such exposing area of the sample compartment, e.g. either by controlling the dimensions of the sample compartment which define the exposing area (such as the walls of the sample compartment), or by forming a mask or and effective window defining the exposing area, either in or on the sample device or in connection with the detection device.

Disposable Device

According to one embodiment, the device of the present invention, can easily be removed from a measuring instrument when a new sample or sample material is to be measured. Apart from allowing a more simple mechanical construction of an instrument used for the collection and analysis of exposed electromagnetic signals, the absence of any permanent flow system in the detection device is advantageous. A further advantage of the device according to the invention is that it can contain the sample in a closed container before, during and after analysis, thus allowing more safe handling of hazardous material.

According to this embodiment, the sample device may be laid out to allow a pre-determined number of determinations to be performed with one device. In this way, a large sample volume may be analysed using one sample device.

One important aspect of the present invention, which is particularly of interest when the sample, or any component added to the sample can be considered hazardous, or difficult to handle, is that it is possible to contain the sample within the device before, during and after the analysis. Prior to analysis, the sample device containing the sample is introduced to a detection system. After the analysis has been performed, the device is readily removed from the detection system, allowing another device to take its place.

Materials

The device is constructed of a material that has the sufficient physical strength as well as being capable of being shaped into the required physical and functional appearance. In particular, the material must be robust during storage, transport and use of the device.

Furthermore, the material must be compatible with the reagents used, in particular reagents pre-arranged in the device, so that the reagent cannot dissolve, react with or diffuse into the material within a predetermined period of time.

Whereas transparency is important for the wall part of the sample compartment where through the signals are passing the transparency of the rest of the device is of less importance, apart from situations where either the sample or a reagent is light sensible even for short exposures to light. Preferably the material contains substantially no fluorescence that would otherwise disturb the assessment.

In particular, a plastic material is useful such as a material selected from polystyrene, polyester, polycarbonate or polyethylene. For other applications, glass is the preferred material for the exposing domain of the sample compartment.

When selecting materials for the sample compartment it is important that the amount of dust and other noise-sources is kept as low as possible. For this reason, it is also preferred to use a flow-through system, which can be washed with a liquid to remove any dust particles which have entered the flow system and the sample compartment during assembly and storage.

The sample device is preferably constructed of a back side and a front side where each side may be moulded individually to be subsequent assembled. The sides of the device are preferably moulded from the same material.

The window area(s) of the sample compartment Is/are preferably moulded separately to be Inserted into the device parts before the final assembly.

Stop Flow Cuvette

In a preferred embodiment of the invention the volume being assessed is substantially at stand-still during analysis, thus allowing the optimal use of measurement time in order to improve any signal to noise conditions. This arrangement also eliminates any error which could be inherent in the assessment of particles caused by variation in flow conditions, particularly when an assessment of a property is a volume related property such as the counting of particles in a volume of sample.

When using a stop-flow cuvette, the detection of signals in step iii) of the method according to the present invention may advantageously be carried out for a period of time, being an exposure time.

The length of the exposure time may be less than 120 sec, for example less than 90 sec, such as less than 60 sec, for example less than 30 sec, such as less than 15 sec, for example less than 5 sec, such as less than 2 sec, preferably less than 1 sec, more preferably less than 0.5 sec, more preferably less than 0.1 sec, more preferably less than 0.01 sec, such as less than 0.001 sec. Compared to flow cytometers, these exposure times are orders of magnitude higher than the typical exposure time. Therefore much more signal can be accumulated in the detection device compared to such prior methods and/or there is less requirement for intense illumination of the particles from external sources.

Preferably during such exposure time the particles move less than a distance corresponding to 150% of their diameter in a direction substantially parallel to the plane of the detection elements. Expressed in another way the particles preferably move less than a distance causing the representation of the particles in the spatial image to move in the image corresponding to 150% of the diameter of the representation of the particle during the exposure time. This can e.g. be obtained by controlling the flow of sample through and/or within the sample compartment during such exposure. More preferably the percentage is less than 100%, preferably less than 75%, for example less than 50%, such as less than 40%, for example less than 30%, such as less than 20%, for example less than 10%.

Flow Through Cuvette

However, in another embodiment, the sample in the sample compartment is moved through the sample compartment during the exposure, and the exposure is performed over a sufficiently short period of time to substantially obtain stand still condition during the exposure. In either case, there is a close control of the volume of the sample from which the exposure is made, which is one very preferred feature of the present invention.

Detection from Different Sub-Volumes

One aspect of the present invention is that more than one portion of the same sample material is subjected to analysis by exposure to the detection system. This can be done by allowing the sample compartment to be moved, thus exposing a different portion of the sample compartment, or by allowing the sample within the sample compartment to flow and thereby substantially replace any sample volume exposed with a different sample volume. The result in both cases is that a new volume of the sample is analysed in the detection device.

Illumination

When at least a major part of the electromagnetic radiation emitted from the sample during exposure originates from or is caused by electromagnetic radiation supplied to the sample from a light source, it is highly preferred that at least a major part of the radiation from the light source having a direction which is transverse to the wall of the sample compartment or a plane defined by the sample compartment (or an increment plane if the compartment wall is curved), or between perpendicular and 10 degrees, preferably between perpendicular and 20 degrees, more preferably between perpendicular and 30 degrees and still more preferably between perpendicular and 45 degrees.

In a preferred embodiment, the backside wall of the sample compartment (i.e. opposite the wall through which the signals are passing) may be provided with a light diffusing effect. This may for example be provided by shaping this window area with a rough surface.

Statistical Requirements, Criteria for Repetition

A central requirement of the present invention is that more than one sub-volume of the same sample is subjected to detection of particles. An important aspect of the invention is the way in which the number of repetitions (examinations of sub-volumes) is chosen.

According to one very simple embodiment the loading and detection steps are repeated a predetermined number of times. This repetition may for example be performed until a predetermined statistical requirement is fulfilled, such as until it can be predicted with a certain degree of likelihood that the sample contains or does not contain a particular particle in an amount below or above a certain threshold value.

Preferably the reliability of the correlation of spatial image data to the number of rare event particles, defined as the probability of identifying a rare event particle in the absence of a rare event particle is less than 33%, such as 20%, preferably less than 20% such as 10%, more preferably less than 10% such as 5%, more preferably less than 5% such as 2%, more preferably less than 2% such as 1%, more preferably less than 1%.

Expressed in another way the reliability of the correlation of spatial image data to the number of rare event particles, defined as the probability of identifying a rare event particle in the precence of a rare event particle is better than 33%, such as 50%, preferably better than 50% such as 75%, more preferably better than 75% such as 90%, more preferably better than 90% such as 95%, more preferably better than 95% such as 99%, more preferably better than 99%.

The steps may also be repeated a number of time until a predetermined volume of sample has been analysed.

Often the predetermined volume of sample is from 10 to 100 μl, preferably from 15 to 25 μl, more preferably approximately 20 μl.

The predetermined volume of samples may also preferably be more than 10 μl, more preferably more than 20 μl, more preferably more than 50 μl, more preferably more than 100 μl.

Another criterion is to repeat the steps until at least one rare event particle has been detected. Thereby it can be said with high certainty that the sample does contain that particular particle.

The absence of a particle is more difficult to ensure with a high degree of statistical certainty. According to this embodiment the steps may be repeated until the absence of a rare event particle has been determined a pre-determined number of times or for a pre-determined sample volume.

The repetitions may comprise serial repetitions in time. In this way the same sample device may be used for all the repetitions. Preferably the device is adapted for use for a certain number of repetitions. It may thus contain sufficient reagents for labelling and staining particles in a certain volume of sample.

According to another embodiment of the invention the repetitions comprise parallel repetitions performed in several sample compartments filled more or less simultaneously with volumes of the same sample.

The steps may in principle be repeated an unlimited number of times if required. Normally, they are repeated at least 3 times, such as at least 4 times, preferably at least 5 times, such as 6, 7 or 8 times, more preferably at least 9 times, such as at least 10 times, for example at least 12 times, such as at least 15 times, for example at least 20 times, such as at least 25 times, for example at least 30 times, such as at least 40 times, for example at least 50 times, such as at least 75 times, for example at least 100 times.

According to most embodiments of the invention, the steps are repeated 20 to 100 times.

As mentioned above, the size of the volume is suitably adapted to the desired statistical quality of the determination. When considering the requirements to the size of the volume of the sample there it is often the nature of the analysis which defines such limits. Often the nature of the sample and the particle of interest in the sample which is to be analysed is one or more of the following:

The presence of a particle in a sample is to be determined, such as the detection of at least one foetal cell in maternal blood. The assessment of the presence of the particle is done in relation to the volume since only rarely the entire sample represents the sample volume.

The absence of a particle in a sample is to be determined, or rather its presence in numbers below a certain low threshold. An example of this application is the analysis of depleted blood.

The frequency of a particle in a sample is to be determined or its presence below a certain frequency threshold.

Detection of Signals

The array of detection elements used for detecting electromagnetic radiation from particles in the sample compartment may comprise a charge coupled device (CCD) or an array of light sensitive diodes such as a CMOS image sensor, preferably a CMOS image sensor with on-chip integrated signal condition and/or signal processing, more preferably a CMOS image sensor with on-chip integrated computing means capable of performing image processing.

The detection of electromagnetic signals may comprise one or more frame grabbing actions. Preferably the detection comprises more than one frame grabbing action such as at least two frame grabbing actions, such as three frame grabbing actions, for example at least four frame grabbing actions, such as five frame grabbing actions, for example at least six frame grabbing actions, such as seven frame grabbing actions, for example at least eight, nine, ten or more frame grabbing actions. The advantage of using several frame grabbing actions is that signals may be averaged and thereby the effect of noise reduced. With the relatively long exposure times typically used according to the present invention, a high number of frame grabbing actions is possible and the signal to noise ration can be increased.

One-Sided and Two-Sided Systems

The following is a description of illumination and detection systems, which may be used in conjunction with the present invention for illuminating and detecting rare event particles in the exposing domain of a sample compartment. The advantage of the described systems is an improved signal to noise ratio.

The detection device may be laid out as a one-sided device, i.e. a device for which the excitation light is directed to the sample from the same side of the sample as the side for which the signals emitted from the sample are detected.

By this apparatus a variety of advantages have been achieved as compared to conventional fluorescence microscopes. First of all it is possible to arrange the sample to be assessed directly in the sample plane instead of sliding it into the sample plane between the detector and the excitation light. Furthermore it has become possible to detect surface fluorescence of a sample not being transparent.

As mentioned above it is also possible to increase the intensity of the excitation light without compromising the detectors.

Also samples having a nature whereby it is normally not possible to arrange the sample in a microscope may be assessed by the use of the present system, in that the microscope may be placed directly on the sample whereby the surface of the sample simply constitutes the sample plane.

Finally it is possible to produce a more compact and thereby more easily handled apparatus, in that the excitation light means is arranged on the same side of the sample plane as the detector, thus shortening the axis of the apparatus by at least 25% as compared to conventional apparatuses.

By the present invention it is possible to assess parameters of a sample which has up to now only been reliably assessed by the use of flow cytometric equipment. It is possible to assess parameters of a large sample in one exposure thus reducing the statistical errors normally counted for when assessing large samples by assessing only parts thereof per exposure.

Furthermore, it is possible to obtain more than one fluorescence signal from the sample in one exposure thereby facilitating classification of particles of the sample, due to their different fluorescence signals.

Thus, the one-sided apparatus according to the invention may be constructed in a wide variety of combination, which are all within the scope of this invention. In particular the principal combination discussed below are envisaged.

The apparatus may be constructed as a single fluorescence apparatus wherein the light sources and the excitation light filters are identical.

A multiple fluorescence apparatus, such as an apparatus providing at least two different fluorescence signals, may be provided by at least one of the following:
A first and a second light source, said light sources emitting light in different wavelengths
A first and a second filter being different whereby the excitation light of at least two different wavelength are exposed to the sample
A first and a second emission filter being different, such as a dual band filter, whereby at least two different fluorescence signals are emitted to the detector(s)

It is however a further advantage that the present apparatus may be constructed as a double-sided apparatus, whereby excitation light may be directed onto the sample from both sides of the samples, or detection means are arranged to detect signals from both sides of the samples, or a combination of both.

Thus by a double-sided apparatus is meant an apparatus according to the invention further provided with:
A second excitation light means located in a second light plane, said second light plane being parallel with the sample plane and located on the other side of the sample plane as opposed to the first light plane. Thereby the sample is receiving excitation light from both sides of the sample considerably increasing the energy exposed to the sample, and/or
A second detection means arranged so that the sample is positioned between the first detection means and the second detection means. Hereby it is possible to assess different information regarding the signals from the sample by one exposure detection. For example the first detection means may be adapted to register the number of particles of the sample, whereas the second detection means is adapted to register the morphology of the particles in the sample.

In a preferred embodiment the double-sided apparatus comprises both double-sided excitation system and double-sided detection system.

The second excitation light means may be any of the light means discussed in relation to the first light means. Depending on the purpose of the fluorescence microscope the light means may be different or identical.

Furthermore, it may be of interest that the excitation light would constitute different wavelength bands whereby illumination with different wavelengths is achieved.

The second detection means may be any of the detection means discussed in relation to the first detection means.

Any suitable combination of light sources, filters, magnification and detectors are envisaged by the present invention. In the following preferred embodiments of the two-sided system is discussed.

The apparatus may be a single fluorescence system, wherein excitation light of substantially identical wavelength are exposed to the sample from two sides. Thereby the excitation light may be intensified.

In a double-sided excitation light apparatus a first excitation light means exposes the sample to one wavelength from one side of the sample, and the second excitation light exposes the sample to another wavelength from the other side of the sample. It is understood herein, that of course the first excitation light and the second excitation light respectively, may comprise different light source and/or filters, whereby the sample may be illuminated with even more wavelengths as discussed above.

The double-sided excitation light apparatus may comprise one detector, whereby the apparatus functions as a partly transmitting system.

In another embodiment the double-sided excitation light apparatus comprises two detecting means. Thereby an increased amount of information may be obtained from the sample. In one aspect the two detecting means may obtain equal, although mirror images (the images on the two detectors are mirror images of each other), information relating to the sample providing a validation of the information.

The apparatus according to the invention may also be a double-sided detection apparatus using a one-sided excitation light means. Thereby one detector detects signals being transmitted through the sample.

Independent of the arrangement of excitation light, a double-sided detecting system is capable of increasing the amount of information received. For example different wavelength may be received by the two detectors, and or different detectors, having different sensibility may be used. Furthermore, by using for example different magnification for the two detectors the information relating to the sample may be increased. One side of the system may assess for example number of particles in a large area of the sample, for example by a low magnification, and the other side of the system may assess the morphology of the particles by using a larger magnification. Combinations of magnification may for example be 1:1 and 1:4, 1:1 and 1:10, 1:2 and 1:4, 1:2 and 1:10. The signal information transferred from the two detectors is preferably transmitted to the same processor, whereby the information may be displayed separately, as well as being combined providing for example specific morphology information related to specific particles the position and number of which are detected by the other detector.

It is also possible to use the apparatus according to the invention as a double-sided apparatus where the other side is a conventional light microscope or any other type of microscope. When using the other side of the system as a non-fluorescence microscope, the illumination light for the microscope may be suitably arranged on either side of the sample in relation to the microscope.

The double-sided apparatus comprising a conventional microscope on one side, may comprises a one-sided or a double-sided excitation light system for the fluorescence part of the system.

When using a double-sided detection system the processor of the first detection means may receive signal data from the second detection means as well in order to simplify the apparatus. It is however possible to install a separate processor for each detection means.

### EXAMPLES OF ONE AND DOUBLE SIDED EXCITATION AND DETECTION SYSTEMS

In the following one embodiment of the detection system is discussed in more detail in relation to the drawings.

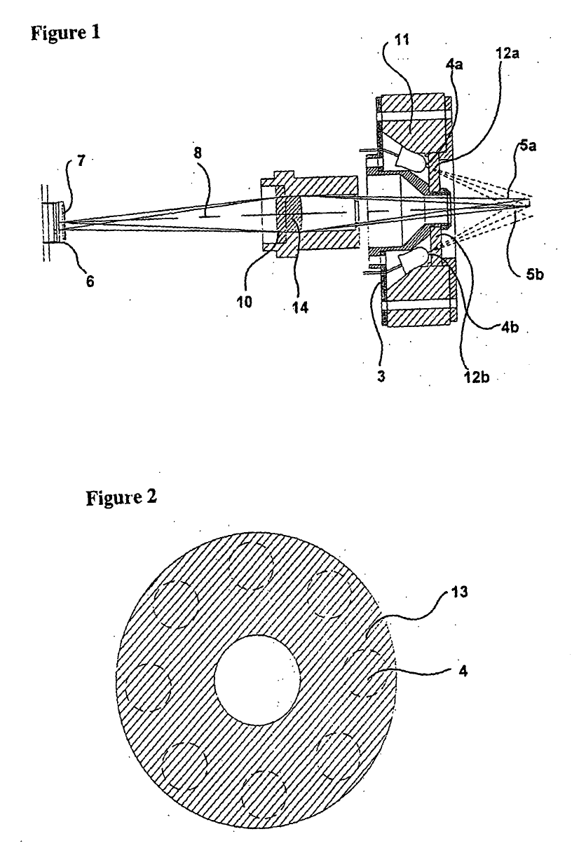

In FIG. 1 an example of the illumination and detection system 1 is shown in schematic form. The sample is arranged in a sample compartment 2 the sample plane. Excitation light from the light sources 4a, 4b in the excitation light means 3 is exposed onto the sample through a main light path 5a, 5b.

Fluorescence signals from the sample is emitted to the detection means 6 comprising at least one detector 7. The path of the emitted signals is following an axis between the sample and the detector, the detection-sample axis 8.

The signal data are transmitted to a processor 9 coupled to the detecting means 6. The fluorescence signals from the sample is filtered by means of emission filter 14 and focused to the detection means 9 by means of a focusing lens 10.

The light sources 4a, 4b are arranged in a light housing 11, whereby the transmission of excitation light directly to the detection means is avoided. Furthermore excitation light filters 12a, 12b are positioned in the excitation light beam.

FIG. 2 shows a cross-section of the circular supporting material 13 of the excitation light filters wherein the position of the light sources have been indicated by circles in broken lines.

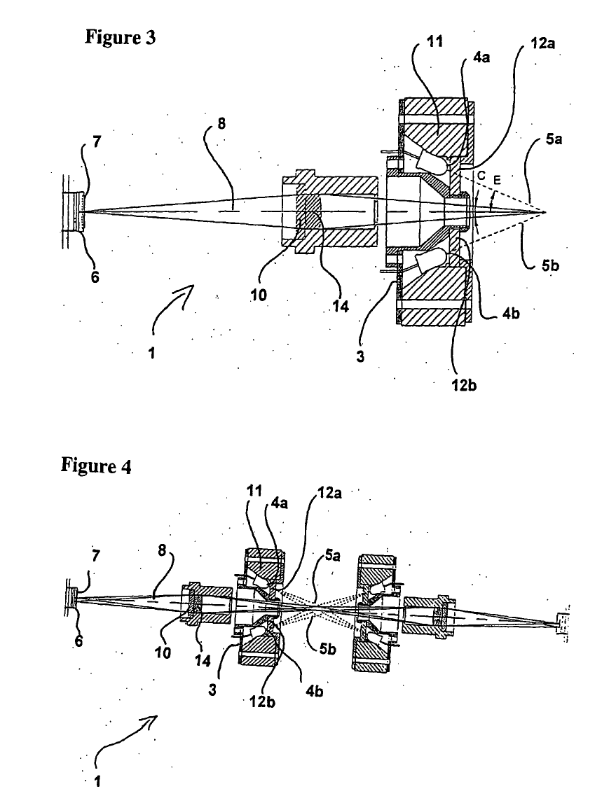

In FIG. 3 the light path and signal path is shown in more detail. In the light path the main light path is shown as 5. Furthermore, the detection-sample axis is shown by broken lines 8. The collection angle of the system is denoted C shown between two arrows and the angle between the main light path and the detection-sample axis is denoted E.

In FIG. 4 a double-sided excitation/detection system 1 is shown wherein the systems on each side of the sample are identical and as described for the one-sided system of FIG. 1.

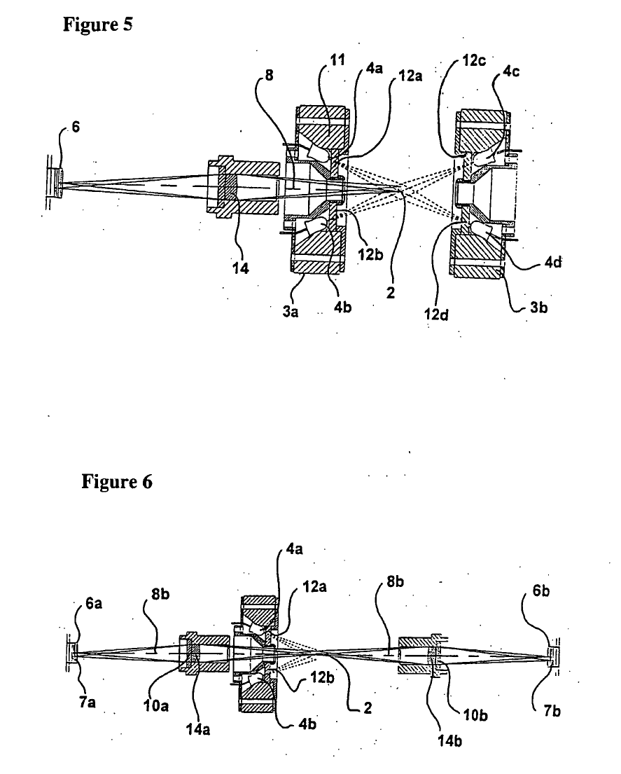

FIG. 5 shows a double-sided excitation system wherein excitation light from the light sources 4a, 4b in the first excitation light means 3a and excitation light from the light sources 4a, 4b in the second excitation light means 3b is exposed onto the sample 2 from both sides of the sample 2. As discussed above, the light sources may be identical or different depending on the information to be assessed. Furthermore, the filters used for each light source may be different or identical.

Fluorescence signals are transmitted through and reflected from the sample due to the excitation light arrangement and emitted to the detection means 6. The path of the emitted signals is following an axis between the sample and the detector, the detection-sample axis 8.

The signal data are transmitted to a processor coupled to the detecting means as described above.

FIG. 6 shows a double-sided detecting system, using a single-sided excitation system, wherein reflected fluorescence signals from the sample 2 are detected by detecting means 6a comprising detector 7a. The reflected fluorescence signals are transmitted though filter 14a and focused by lens 10a.

Furthermore, transmitted fluorescence signals from the sample 2 are detected by detecting means 6b comprising detector 7b. The reflected fluorescence signals are transmitted though filter 14b and focused by lens 10b.

Filter 14a is preferably different from filter 14b, whereby information relating to at least two different fluorescence signals is obtainable.

Also the magnification in the two detecting systems may be different, for example by lens 10a being different from lens 10b.

System for Isolation of Rare Event Particles

The invention also features a system for collection and a system for isolation of a rare event particle. The particle may for example be isolated from other non-rare particles in the same sample.

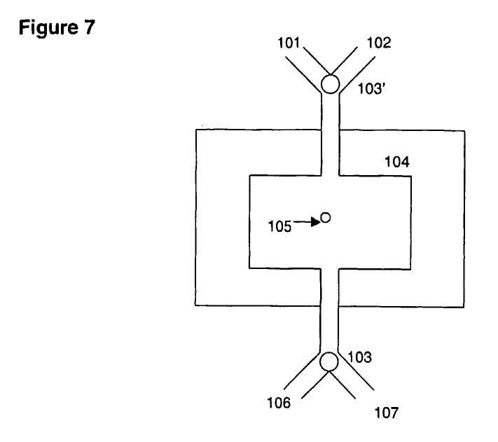

The simplest version of the two systems is the system for collection of a rare event particle. A schematic example of such system is shown in FIG. 7. The system has a sample inlet (101), which leads sample to the sample compartment (104). Once a sample volume with a rare event particle (105) is inside the sample compartment, the presence of the rare event particle (105) is detected and the volume containing the particle is flown from the sample compartment, either by leading a carrier liquid through another inlet (102) or by replacing the sample in the sample compartment with new sample. In any event the sample with the rare event particle(s) is directed to a rare event particle tube (106). Sample volumes not containing any rare event particles are flushed through the waste tube (107) to a waste container. A valve is placed on the outlet side (103) to control the flow of sample and to direct waste and rare event particle liquid to the two different outlets (106, 107). The system may also comprise a similar valve (103′) on the inlet side.

The system for isolation of a rare event particle contains all these features and in addition on the inlet side a carrier liquid inlet (102) and a valve (103′). When the presence of a rare event particle in the exposing domain is detected, the sample with rare event particle(s) is flushed to the rare event particle outlet (106) using a carrier liquid, which flushes and dilutes the sample. Sample without rare event particle(s) are flushed to the waste outlet. The diluted samples with the rare event particle(s) may then be entered into the exposing domain again to further separate the rare event particle(s) from other particles. Through successive rounds of detection and dilution the rare event particles end up being substantially the only particles in the sample.

Through the action of washing the rare event particle from the sample compartment, the sample volume with the rare event particle is diluted with the carrier liquid. This diluted sample liquid can then be re-entered into the sample compartment through inlet 101. The diluted rare event particle liquid is then re-analysed and any sub-volumes not containing the rare event particle are flushed to the waste. By running the sample with the rare event particle through the sample compartment a number of times other particles in the sample are removed stepwise resulting in a volume, which substantially only contains the rare event particle.

Preferably the detection of absence or presence of a rare event particle is performed according to the method described in the present invention.

According to one embodiment of the isolation method the exposure time during the initial steps of isolation are shorter than during the later steps of isolation. When using this method the precision in the later steps of isolation is increased.

Advantageously the method comprises filtration of the sample comprising the isolated rare event particle and diluted with carrier liquid, to reduce the volume of sample in which the rare event particle is present or to retain the rare event particle or a filter.

Preferably the system further comprises tube means to connect the rare event particle tube (106) on the outlet side to the sample inlet (101).

Preferably the exposing domain of the system comprises a precisely defined volume of sample in the exposing domain comprises 0.1 to 1000 μl, such as from 1 to 50 μl, for example from 2 to 20 μl, such as from 3 to 10 μl; from 50 to 100 μl, or from 100 to 150 μl, or from 150 to 250 μl, or from 250 to 350 μl, or from 350 to 500 μl, or from 500 to 750 μl, or from 750 to 1000 μl.

The system may further comprise detection means comprising an array of detection elements on which a spatial Image of the rare event particle(s) in the exposing domain can be formed, as well as a data processor to process the detected images. 60. The array of detection elements may for example comprise a charge coupled device (CCD) or an array of light sensitive diodes such as a CMOS image sensor, preferably a CMOS image sensor with on-chip integrated signal condition and/or signal processing, more preferably a CMOS image sensor with on-chip integrated computing means capable of performing image processing.

According to an especially preferred embodiment of the invention, the system comprises means to detect signals for a period of time, being an exposure time. The means may e.g. be a timer.

The timer may be adapted to allow an exposure time of less than 120 sec, for example less than 90 sec, such as less than 60 sec, for example less than 30 sec, such as less than 15 sec, for example less than 5 sec, such as less than 2 sec, preferably less than 1 sec, more preferably less than 0.5 sec, more preferably less than 0.1 sec, more preferably less than 0.01 sec, such as less than 0.001 sec.

As described in the method above, the precisely defined volume of the exposing domain may be defined in one dimension by walls.

Preferably the precisely defined volume of the exposing domain is in one dimension defined by walls being substantially parallel to the plane of the detection elements and the area viewed by the detection elements.

Alternatively the precisely defined volume of the exposing domain is defined by walls being substantially parallel to the plane of the detection elements and a mask defining an area to be viewed by the detection elements, preferably where the mask is effectively defined by the area which is projected onto the active area of the array of detection elements, preferably where the projection is formed by optical means such as one or several lens(es). The mask may be located on the detection device and/or on the sample device.

The detection of electromagnetic signals may comprise one frame grabbing action or at least two frame grabbing actions, such as three frame grabbing actions, for example at least four frame grabbing actions, such as five frame grabbing actions, for example at least six frame grabbing actions, such as seven frame grabbing actions, for example at least eight, nine, ten or more frame grabbing actions.

At least two of the grabbed frames may be averaged preferably to reduce the electronic noise.

In order to reduce the amount of volume to be re-entered into the sample compartment, the system may comprise means to filter a liquid sample comprising one rare event particle diluted with carrier liquid, while retaining the rare event particle.

Preferably the system further comprises at least one source of illumination to illuminate the sample in the exposing domain. The source of illumination may for example comprise light emitting diodes (LED), lasers, laser diodes, thermal light sources, gas discharge lamp, stroboscopic light or the one or double sided excitation system described above.

Application Directed to Analysis of Rare Event Particles in Blood

In the following, when reference is made to blood, it is intended to encompass the following terms under this definition: Any type of blood or liquid blood fraction from an animal, preferably from a mammal, such as from a human being. Blood, plasma, depleted blood, donor blood, blood fraction, serum, blood product, anti-coagulated whole blood (AWB), packed red cells obtained from AWB; platelet-rich plasma (PRP) obtained from AWB; platelet concentrate (PC) obtained from AWB or PRP; plasma obtained from AWB or PRP; red cells separated from plasma and resuspended in physiological fluid; and platelets separated from plasma and resuspended in physiological fluid.

Depleted Blood, Background

In recent years, in the field of blood transfusion, a leukocyte-free blood transfusion in which leukocytes are removed from a blood product before transfusion is increasingly employed. This is because it has become apparent that side effects of transfusion, such as headache, nausea, chills and non-hemolytic feverish reaction, and side effects more serious to a recipient, such as allosensitization, post-transfusion GVHD (graft versus host disease) and viral infection, are mainly caused by leukocytes contained in a blood product employed in transfusion.

It is known that the number of leukocytes injected into a recipient at one transfusion must be limited to about 100,000,000 or less in order to avoid relatively slight side effects, such as headache, nausea, chills and fever. For meeting this requirement, leukocytes must be removed from a blood product to a level of 10⁻¹ to 10⁻² or less in terms of a leukocyte residual ratio. With respect to allosensitization, it now attracts the greatest attention in the art of blood transfusion, and it is one of the side effects, the prevention of which is most desired. For preventing this serious side effect, it is believed that the number of leukocytes injected into a recipient at one transfusion must be limited to 5,000,000 or less, preferably 1,000,000 or less. For meeting this requirement, leukocytes must be removed from a blood product to a level of 10⁻⁴ or less in terms of a leukocyte residual ratio. With respect to post-transfusion GVHD and viral infection, no generally accepted standards for leukocyte-removal have been established. However, it is expected that infection with a virus, which is believed to exist only in leukocytes, such as cytomegalo virus, adult T cell leukemia virus and post-transfusion GVHD, could be prevented by removing leukocytes to a level of 10⁻⁴ to 10⁻⁶ or less in terms of a leukocyte residual ratio. Further, it is also expected that the probability of infection with a virus, which is believed to exist in both leukocytes and plasma, such as HIV, can be decreased by removing leukocytes.

The methods for removing leukocytes from a blood product can generally be classified into two methods. One is a method in which leukocytes are separated by a centrifuge, taking advantage of a specific gravity difference there between. The other is a filtering method in which leukocytes are removed by a filter comprising a fibre material or a spongy structure as a filter medium. In particular, a filtering method in which leukocytes are adsorption-removed by a non-woven fabric is widely employed due to the advantages of high capability to remove leukocytes, ease in handling and low cost.

It has been the practice for 50 years or more to transfuse whole blood, and more recently blood components, from one or more donors to other persons. With the passage of time and accumulation of research and clinical data, transfusion practices have improved greatly. One aspect of current practice is that whole blood is rarely administered; rather, patients needing red blood cells are given packed red cells (hereinafter PRC), and patients needing platelets are given platelet concentrate. These components are separated from whole blood by centrifuging, the process providing, as a third product, plasma, from which various other useful components are obtained. In addition to the three above-listed components, whole blood contains white blood cells (known collectively as leukocytes) of various types, of which the most important are granulocytes and lymphocytes. White blood cells provide protection against bacterial and viral infection.

In the mid to late seventies, a number of investigators proposed that granulocytes be separated from donated blood and transfused into patients who lacked them, for example, those whose own cells had been overwhelmed by an infection. In the resulting investigations, it became apparent that this practice is generally harmful, since patients receiving such transfusion developed high fevers, had other adverse reactions, and often rejected the transfused cells. Further, the transfusion of packed cells or whole blood containing donor leukocytes can be harmful to the recipient in other ways. Some of the viral diseases induced by transfusion therapy, e.g., Cytomegaloviral Inclusion Disease, which is a life threatening infection to newborns and debilitated adults, are transmitted by the infusion of homologous leukocytes. Another life-threatening phenomenon affecting immunocompromised patients is Graft versus host disease (GVH); a disease in which the transfused leukocytes actually cause irreversible damage to the blood recipient's organs including the skin, gastrointestinal tract and neurological system. More recently, retroviruses such as HIV (AIDS) and HTLV1 have become a threat. Since some viruses, including several of those described above, are resident in the leukocytes, the removal of leukocytes is regarded as beneficial.

Conventional red cell transfusions have also been indicted as adversely influencing the survival of patients undergoing surgery for malignancy of the large intestine. It is believed that this adverse effect is mediated by the transfusion of agents other than donor red blood cells, including the donor's leukocytes.

In the currently used centrifugal methods for separating blood into the three basic fractions (packed red cells, platelet concentrate, and plasma), the leukocytes are present in substantial quantities in both the packed red cells and platelet concentrate fractions. It is now generally accepted that it would be highly desirable to reduce the leukocyte concentration of these blood components to as low a level as possible. While there is no firm criterion, it is generally accepted that many of the undesirable effects of transfusion would be reduced if the leukocyte content were reduced by a factor of about 100 or more prior to administration to the patient. This approximates reducing the total content of leukocytes in a single unit of PRC (the quantity of PRC obtained from a single blood donation) to less than about 1*10⁷. Recently it has become more widely perceived that in order to prevent viral infection by transfused blood, factors of reduction should be more than 100, preferably more than 1000, and more preferably 30,000 or 100,000 fold or more, such as 1,000,000 fold.

After filtering of the blood to remove leukocytes the blood must be analysed to verify that the number of leukocytes has been reduced to the level desired. This is typically done by removing a small sample of depleted blood and counting one or more volumes in a haemocytometer or in a flow cytometer. The first method is laborious since relatively large amounts of blood sample must be analysed to get an estimate of the number of leukocytes. The flow cytometry method is faster, but the signal to noise ratio of flow cytometers is not adapted for the detection of rare events. In flow cytometers the rate of detection may be 5,000 to 10,000 events per second. Therefore only a very small amount of electromagnetic radiation may be picked up from each leukocyte as it passes the detector means and it may therefore be difficult for the detection means to distinguish between the background signal and the signal from a rare event particle.

Quality Control of Blood

In another aspect, the invention relates to a method for quality control of blood comprising
i) bleeding blood from an individual,
ii) arranging a sample of the blood in an exposing domain of a device allowing electromagnetic radiation from cells comprised in a precisely defined volume of at least 1 μl of the blood sample to pass to the exterior,
iii) arranging the sample device in relation to a detection device,
iv) detecting electromagnetic signals from the sample in the exposing domain by forming spatial images of the particles on an array of detection elements in the detection device, the ratio of the image of a linear dimension on the array of detection elements to the original linear dimension in the exposing domain being smaller than 10:1, and
v) correlating the detected signals to at least one parameter of the blood.

The method may be performed on un-fractionated blood, but according to a preferred embodiment, the method further comprising fractionation of the blood into blood fractions prior to arrangement of the sample in the exposing domain.

The fractions may comprise whole blood, plasma, depleted blood, donor blood, serum, blood product, anti-coagulated whole blood (AWB), packed red cells obtained from AWB; platelet-rich plasma (PRP) obtained from AWB; platelet concentrate (PC) obtained from AWB or PRP; plasma obtained from AWB or PRP; red cells separated from plasma and resuspended in physiological fluid; and platelets separated from plasma and resuspended in physiological fluid.

According to some aspects, the method may further comprise repeating steps ii) to iv) until a pre-determined statistical requirement is fulfilled. This is the preferred method when the frequency of occurrence of the particle(s) to be detected is very low, such as when the blood has been depleted to remove the majority of leukocytes.

According to some aspects, steps ii) to iv) may be are repeated until one event has been detected. This is the preferred method, when the method is directed to detection of foetal cells in a blood sample from a pregnant woman or animal.

Expressed in a more simple way the steps ii) to iv) may be repeated a pre-determined number of times or they may be repeated a number of times until a predetermined volume of sample has been analysed. This is another way of expressing, that the steps are repeated until a certain statistical requirement is fulfilled.

The determination of a parameter relating to blood, may comprise the detection of absence of an event and/or particle is detected every time. One example of this application is the analysis of depleted blood, whereby the absence of leukocytes is detected every time, preferably until a certain, predetermined volume of depleted blood has been examined.

The individual, from whom blood is bled, may be a blood donor or a patient.

The quality parameter may be selected from the group comprising a differential leukocyte count, HIV detection, hepatitis B, hepatitis C, the level of CD4 lymphocytes, malaria, sickle cell anaemia.

The quantity parameter may be selected from the group comprising a whole blood cell count, a leukocyte count, an erythrocyte count, a platelet count.

The method may be performed at any time in relation to the bleeding. Thus the quality control may be performed substantially during bleeding of the individual. Thereby, information pertaining to the blood is obtained almost instantaneously or shortly after bleeding of the blood.

Alternatively or additionally the steps ii) to v) may be performed after bleeding of the individual. This could be as a quality control of the blood before fractionation and further treatment of the blood. Detection of the quality or quantity parameter(s) after bleeding may also be in connection with diagnosis of an individual, e.g. in a clinic or hospital.

The quality control comprising steps ii) to v) may be performed after storage of the blood, e.g. after storage of a portion of donor blood. One advantage of this embodiment of the invention, is that the quality of the blood can be determined both during bleeding, after bleeding and after storage to ensure that the quality of the blood fulfils the quality requirements of the intended use. Another example is quality control of blood, when the control is performed in a central laboratory and the collection of blood or blood samples is performed in clinics or on farms.

Similarly, the quality control comprising steps ii) to v) may be performed in connection to or prior to infusion of the blood or blood fraction into a patient. In this way a higher degree of certainty concerning the quality or quantity parameter(s) is obtained immediately prior to or even during infusion of the blood or blood fraction.

According to an especially preferred embodiment the blood is collected in a blood collection means and the result of a correlation of step v) is printed on a label on the blood collecting means by the detection device or by a printer connected to the detection device. This embodiment ensures increased certainty in the pairing of blood samples or blood bags with analytical results relating to the blood sample, donor blood, or blood fraction.

The sample of blood, which is to be arranged in the exposing domain may be taken from a blood bag, a blood bag set, or from a tube connected to a blood bag or a blood bag set.

Preferably, the sample device used for quality control comprises a device according to the present invention.

Depleted Blood

In a further aspect, the invention relates to a method for preparation of depleted blood comprising the steps of
i) passing blood through a filter to a blood bag or to a blood bag set and lowering the amount of white blood cells by more than 100, preferably more than 1000, and more preferably 30,000 or 100,000 fold or more, such as 1,000,000 fold,
ii) arranging a sample of the blood in an exposing domain of a sample device allowing electromagnetic radiation from cells comprised in a precisely defined volume of at least 1 μl of the blood sample to pass to the exterior,
iii) arranging the sample device in relation to a detection device,
iv) detecting electromagnetic signals from the sample in the exposing domain by forming spatial images of the particles on an array of detection elements in the detection device,
v) repeating steps ii) to iv) at least once for new volumes of blood sample,
vi) correlating the spatial image information to a quality parameter of depleted blood.

The quality parameter to be assessed after filtration of the blood may comprise the number of white blood cells per volume unit, and/or the ratio of white blood cells to red blood cells or to all blood cells, and/or the percentage of remaining white blood cells. In order to calculate these parameters, it is necessary to determine the number of white blood cells per volume unit, optionally before and after filtering and optionally the number of red blood cells per volume unit.

The sample of blood which is examined in the sample device may be taken from the blood bag or blood bag set or from a tube connected to the blood bag or blood bag set and transferred to an independent sample device. According to another embodiment, the sample device is an integrated part of the blood bag, the blood bag set or a tube connected thereto and the blood sample may be transferred to the sample device by the activation of a valve and/or a pump and/or similar means adapted to draw or force the sample into the sample device.

Often, the method further comprises ascertaining that the number of white blood cells is below a pre-determined threshold. For many applications, medical authorities have determined a threshold value for transfusion blood and/or for depleted blood to be used for transfusion and/or for the manufacture of blood products. It is important to be able to verify, that the method used for depletion of blood results in the desired quality.

The threshold value may be 10,000 particles per ml of sample liquid or lower, more preferably less than 1,000 particles per ml of sample liquid, more preferably less than 100 particles per ml, for example less than 10 particles per millilitre of sample liquid, such as less frequently than 4 particles per millilitre.

The volume of blood sample from which electromagnetic radiation passes to the exterior is at least 2 μl, such as at least 3 μl, for example at least 4 μl, such as at least 5 μl, for example at least 7.5 μl, such as at least 10 μl, for example at least 15 μl, such as at least 20 μl, for example at least 25 μl, such as at least 50 μl, for example at least 100 μl. The volume of blood being examined in one exposure should be as large as possible, since the examination relates to the detection of rare events. In principle there is no upper limit. In practise, an upper limit is determined by the size of array of detection elements used for detection of

By increasing the volume of blood sample which may be examined by one exposure it is possible to lower the number of the number of times that steps ii) to iv) should be repeated.

One of the characteristics of the present invention is that relatively small magnification is used. By keeping the magnification small, more electromagnetic radiation can be accumulated for each time unit per blood cell examined and a better focus depth is obtained. Therefore less externally supplied radiation is required and there is less chance of overheating the sample and less requirement for cooling of the sample compartment. The ratio of the image of a linear dimension on the array of detection elements to the original linear dimension in the exposing domain is preferably smaller than 100:1 (corresponding to a linear enlargement of 100×). But other ratios are also possible such as smaller than 40:1, for example smaller than 10:1, such as smaller than 5:1, for example smaller than 2:1, such as smaller than 1:1. The optimum choice of ratio depends to a large extent on the size and effectiveness of the detection elements used for detecting electromagnetic radiation from the white blood cells. The preferred the ratio of a linear dimension of the image on the array of detection elements to the original linear dimension in the exposing domain is in the range from 10:1 to 1:10. More preferably, the ratio of a linear dimension of the image on the array of detection elements to the original linear dimension in the exposing domain in the range from 1.5:1 to 1:2.

A detection of electromagnetic radiation from the blood sample may comprise one or more exposures. Averaging of results from several exposures may be used to increase the signal to noise ratio by lowering the background signal. Background signal is likely to vary randomly around 0. Averaging the background over two or more exposure periods will cause the signal from background to approach 0.

A detection comprises at least 3 exposures, such as at least 4 exposures for example at least 5 exposures, such as at least 10 exposures, for example at least 15 exposures, such as at least 25 exposures, for example at least 50 exposures, such as at least 100 exposures, for example at least 200 exposures, such as at least 500 exposures, for example at least 1000 exposures. A very high number of exposure periods may be used in conjunction with stroboscopic illumination of the blood sample in the sample compartment.

The duration of at least one exposure may comprise at least at least 0.1 second, more preferably at least 0.5 sec, such as at least 0.7 sec, for example at least 1 sec, for example at least 1.5 sec, such as at least 2 sec, for example at least 3 sec, such as at least 4 sec, for example at least 5 sec, such as at least 10 sec, for example at least 20 sec, such as at least 30 sec, for example at least 40 sec, such as at least 50 sec, for example at least 60 sec, such as at least 90 sec. for example at least 120 sec.

The steps ii) to iv) of the current method for preparation of depleted blood may be repeated a number of times until a pre-determined statistical requirement is fulfilled.

Alternatively or additionally the steps may be are repeated until one event has been detected. This will correspond to detecting one white blood cell in a volume of blood, the total volume examined increasing every time a new sample is arranged in the sample compartment.

According to another embodiment of the method, the steps ii) to iv) may be repeated a pre-determined number of times. The pre-determined number of times may correspond to examining a predetermined volume of depleted blood.

According to another embodiment, the method comprises repeating steps ii) to iv) a number of times until a predetermined volume of sample has been analysed such as from 10 to 100 μl, more preferably from 15 to 25 μl, such as approximately 20 μl.

The method may comprise detecting the absence of an event and/or particle is detected every time.

Preferably, the method comprises the use of a device with a sample as herein described. More preferably, the device comprises a blood bag or a blood bag set with integrated filtering means for selectively removing white blood cells.

In order to obtain blood with an even higher degree of depletion, the method advantageously comprises at least one further filtration step, such as at least two further filtration steps, for example at least three further filtration steps, such as at least four further filtration steps, for example at least five further filtration steps, whereby the amount of white blood cells in each step is further reduced by more than 100, preferably more than 1000, and more preferably 30,000 or 100,000 fold or more, such as 1,000,000 fold. After each filtration step, the number of remaining white blood cells may be determined using steps ii) to vi).

The serial arrangement of sample, detection of radiation, and re-arrangement of sample may be carried out in a stop flow cuvette, wherein a sample volume is introduced, the flow is stopped while radiation is detected by the detection elements, and the sample is replaced by a new sample volume.

According to another embodiment of the invention, a flow cuvette may be used, in which blood sample is continuously flowing and radiation is detected using short exposure times to obtain substantial stand-still condition of the cells in the depleted blood.

### Example 1a

### A System Suitable for the Detection and Assessment of Rare Particles

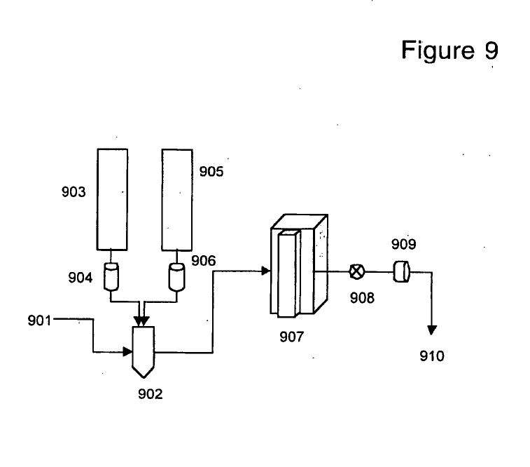

A system suited for the detection and assessment of rare particles, based on the interaction of electromagnetic radiation with the particles, such as absorption or fluorescence is illustrated schematically in FIG. 9.

The figure illustrates a system, where the sample 901 is added to a sampling compartment and mixed with reagent 903, preferably where the volume of the reagent is controlled with a pump 904 capable of substantially precisely measuring a predetermined volume of the reagent.

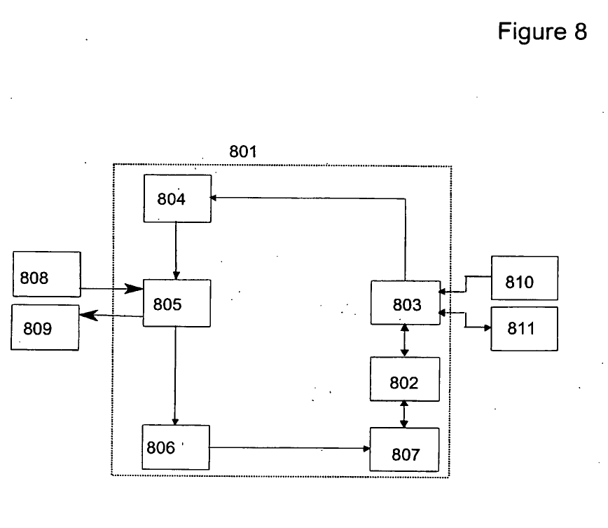

After mixing the sample/reagent mixture is transported into the detection unit 907 by the use of a pump 909. The counting unit is illustrated schematically in FIG. 8.

FIG. 8 illustrates many of the important units and/or operations of a detection unit 801. Most of the controlling of the counting unit, and preferably means to perform analysis of the spatial image are located in the main control unit 802. The main control unit can interact with excitation light source 803 capable of illuminating the sample with light. This light can be focused and/or spectrally modified in an optical unit 804. A typical spectral modification could be selective removal of one or several wavelength elements in the excitation light.

The main controlling unit is also connected to the detection unit 807, usually equipped with one or more sensor, sensitive to electromagnetic radiation. Often it is desired to focus and/or spectrally modify any light entering the detection unit. This can be done with the optical unit 806.

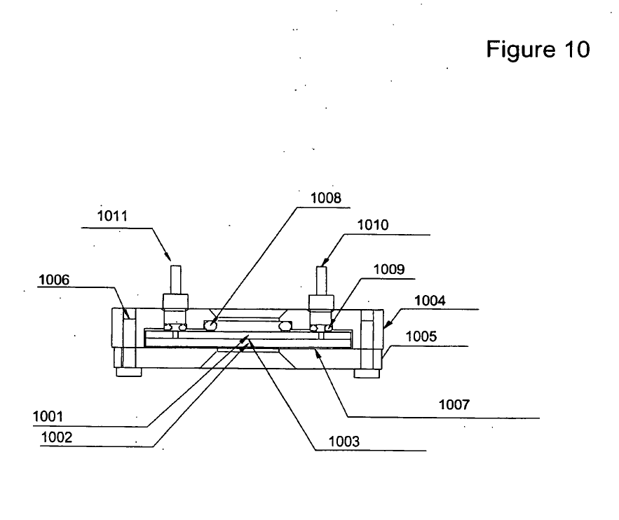

The sample or the sample mixture is introduced to the detection unit through a sample inlet 808, and normally the sample or the sample mixture is removed through a sample outlet 809. During detection at least a fraction of sample or sample mixture is placed in a sample compartment 805 which is further illustrated schematically in FIG. 10.

FIG. 10 illustrates a suitable sample compartment to be used in a detection unit. Two walls of the detection unit are formed by windows 1001 and 1002. These windows are separated by a membrane or a spacer forming a predetermined and/or a determinable distance between the two glass windows. The window sandwich is held together by two mechanically stable parts 1004 and 1005, preferably of metal or plastic. Part 1004 and/or part 1005 are formed with an area, preferably at or near the centre of the part where electromagnetic radiation can enter the sample through the windows. Parts 1004 and 1005 are held together with means 1006 and a suitable pressure on the window sandwich is maintained by the flexible means 1007 and/or 1008. This pressure is preferably such that the sandwich can withstand a pressure which normally occurs when the sample compartment is filled through the sample inlet 1010 fitted securely to the inlet of the window sandwich by fitting means 1009. The sample can leave the sample compartment through sample outlet 1011.

The flow through the detection unit can be controlled by a valve 908 and upon completed analyis the sample can be directed to waste through the outlet 910.

### Example 1b

### System for the Detection and Assessment of Rare Particles

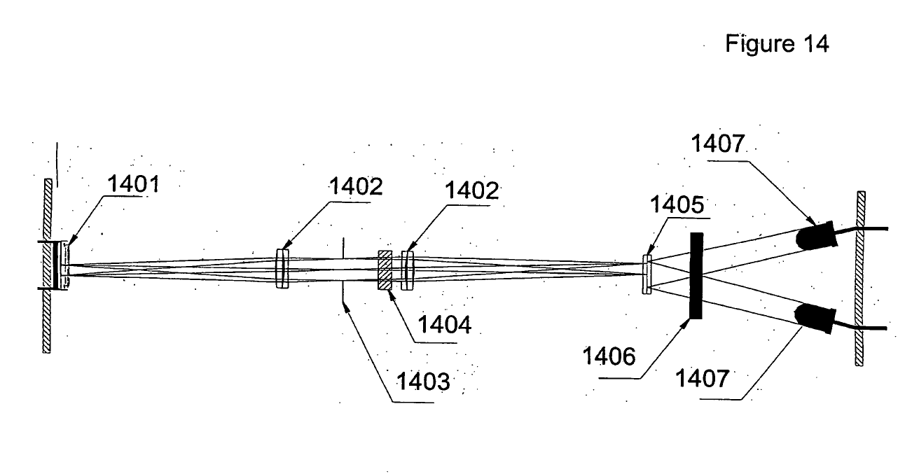

FIG. 14 illustrates a suitable optical system for performing such analyses within the detection unit 907. 1401 is a CCD that captures the image. 1402 is an achromatic glass lenses used for imaging the volume inside the sample compartment to the CCD. The position of the lenses along the optical axis determines the transversal magnification and is used for focusing the system. 1403 is an aperture. 1404 is a glass emission absorbance filter, allowing substantially only red fluorescent light to pass. 1405 is the sample compartment being in close proximity to the optical system object plane. 1406 is the glass excitation interference and absorbance filter, allowing substantially only green excitation light to pass. 1407 is the Light Emitting Diodes (LED) working as a light source in the optical system emitting green light at the wavelength of around 530 nm.

The volume of sample for each measurement is approximately 3 μL (140 μm spacer×approx 22.0 mm²) and the total sample per analysis (20 measurements at 2:3 dilution of sample) is then approx. 40 μL. This volume can be adjusted to higher volumes (e.g. 60 or 100 μL sample per analysis) by increasing the number of measurements thereby increasing the sensitivity of the system further.

The excitation filter is a combined interference and absorbance 550 nm short wave pass filter with an additional infra red (IR) blocking layer. The excitation filter is also anti-reflection coatied on the one side that faces the light source. The emission filter is an absorbance 590 nm long wave pass filter.

The light source is light emitting diodes (LED) with a spectral peak about 517 nm, type Nischia NSPG500S.

The Transversal Magnification (MT) is approximately 0.92. The Numerical Aperture (NA) is approximately 0.05. Both imaging lenses are low-cost achromatic lenses having diameter of 9 mm and focal lenght (FL) of 50 mm.

The length of the optical system from the object plane to the CCD plane is approximately 130 mm and the total lenght of the detection unit is approximately 170 mm including the component print circuit boards.

Object size discrimination: 10 pixels. Only objects equal to or smaller than 10 pixels are being identified as cells. Exposure time per image is 0.5 sec. PC type: Dell Inspiron2500 (Laptop with Intel Pentium III, 800 MHz, 128 Mb RAM, with USB port) PC software: Operative system: Microsoft Windows2000 (Microsoft), Application software: LabVIEW v.6i (National Instruments, Texas USA), Image depth: 8 bit (256 greyscale colors). CCD type: SONY ICX404AL, 510×492 pixels, interlaced readout (images are 510×246 pixels). CCD physical size: 4.96 mm×3.69 mm (4:3 scale).

### Example 2

### Comparison of the Performance of a System According to the Present Invention and a Commercially Available System

10 samples of leukodepleted Red Blood cell units (SAGM blood units), prepared from human blood were analysed with a method according to the present invention and the results compared to a commercially available method suitable for the analysis.

For the measurement by a method according to the present invention a volume of 300 μl of the sample was added to 150 μl reagent. The reagent consisted of Propidium iodide (25 μg/ml), Triton X-100 (1.5% w/w) and polymer surfactants Pluronic (1.0% w/w) dissolved in water. The sample reagent solution was mixed by pipetting prior to analysing in the detection unit. A total of 20 images were analysed and the number of observed rare particles were thus counted in a volume corresponding to approximately 20 μl of the initial sample. This analysis were carried out twice rendering duplicate results for each sample with the system described in Example 1b.

A portions of the same samples were analysed according to the LeukoCount method (Becton Dickinson).

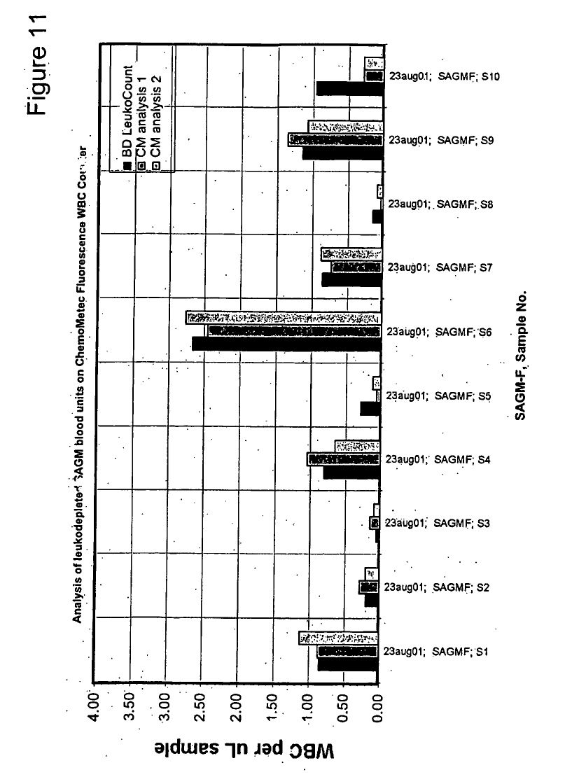

The results of the analysis (expressed in White Blood cell Count per μl) are given in the following table and illustrated in the graph in FIG. 11.

| LeukoCount | CM 1 | CM 2 |
|---|---|---|
| 0.85 | 0.87 | 1.12 |
| 0.19 | 0.28 | 0.20 |
| 0.05 | 0.14 | 0.08 |
| 0.80 | 1.04 | 0.65 |
| 0.28 | 0.06 | 0.11 |
| 2.65 | 2.4 | 2.75 |
| 0.85 | 0.73 | 0.87 |
| 0.14 | 0.03 | 0.08 |
| 1.14 | 1.35 | 1.07 |
| 0.95 | 0.28 | 0.28 |

The conclusion which can be drawn from the results is that there is a good correlation between the two methods. This suggest that both methods estimate the same analyte.

### Example 3

### Repeatability of Various Methods for the Assessment of WBC in Leukodepleted Blood or Blood Products

The literature reports the Coefficient of Variation (CV) of the repeatability of different methods. This has been compared to the estimated CV for a method according to the present invention.

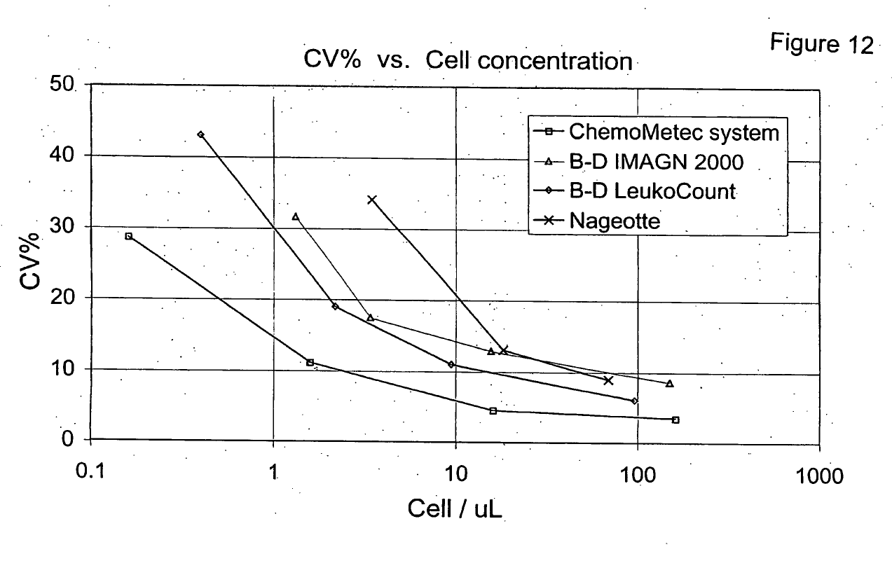

When estimating the WBC in leukodepleted blood or blood product the three routinely used methods is plotted in the graph shown in FIG. 12. These methods are:
IMAGN 2000 instrument (Becton Dickinson)
LeukoCount method on flow cytometer (Becton Dickinson)
Nageotte method (manual microscopy method)

IMAGN 2000 instrument data are from Becton Dickinson sales material 1999.

LeukoCount method on flow cytometer data are from Becton Dickinson sales material 1999.

Nageotte data as presented on ISBT, Jul. 15-18, 2001, Paris, France by P. F. van der Meer and from Clin. Lab. Haem. Vol. 23, p 43-51, 2001.

For comparison tests were conducted according to a method of the present invention and the results are give in the graph in FIG. 12.

The conclusion from the obtained results suggest that the Coefficient of Variation of a method according to the present invention is not higher, and probably lower than the reported CV of other routinely used methods.

### Example 4

### The Effect of Polymer Surfactants on the Sensitivity of a Method According to the Present Invention

Often the rare particle being analysed is a biological molecule or a biological particle. Such particles can often have tendency to interact physically or chemically with particles or surfaces. In the present example the effect of the use of polymer surfactants on the assessment is illustrated.

An experiment was carried out in accordance with the settings of Example 2 with the exception that two reagents were used, one containing the polymer surfactant Pluronic and the other without the addition of any polymer surfactant.

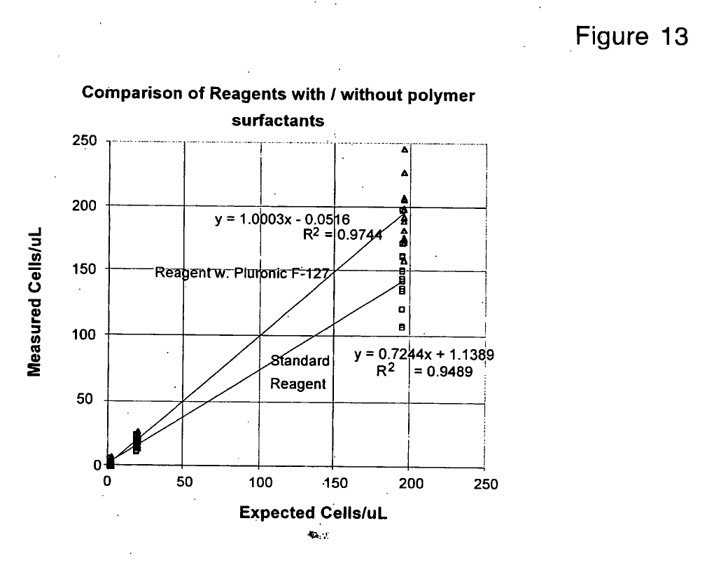

Three samples of leukodpeleted blood product were measured according to a method of the present invention. Each sample was measured in 15 replicates with each of the two reagents. The result is given in the graph in FIG. 13. The graph shows measured WBC vs. the expected WBC when using the two different reagents.

The conclusion which can be drawn from the results is that approximately 25% higher sensitivity was observed under the conditions used in this experiment.

1-83. (canceled)

84. A method for detecting a rare event particle in a liquid sample comprising the steps of:
i) in a sample device arranging a precisely defined volume of at least 0.1 μl of a liquid sample in an exposing domain of a sample compartment, allowing electromagnetic radiation from the rare event particle(s) in the exposing domain to pass to the exterior,
ii) arranging the sample device in relation to a detection device so that signals from the exposing domain can pass to an array of detection elements in the detection device,
iii) detecting electromagnetic signals from the first volume of liquid sample in the exposing domain by forming a spatial image of the rare event particle(s) on the array of detection elements,
iv) repeating steps i) and iii) at least once for new volumes of the same liquid sample,
v) correlating the spatial image to the number of rare event particle(s) in the volume of liquid sample in the exposing domain.

85. The method according to claim 84, where steps i) and iii) are repeated a predetermined number of times.

86. The method according to claim 84, where the steps are repeated a number of times until a predetermined statistical requirement is fulfilled.

87. The method according to claim 84, wherein the probability of the occurrence of at least one exposure without any particles this probability is at least 2%.

88. The method according to claim 84, where the reliability of the correlation of spatial image data to the number of rare event particles, defined as the probability of identifying a rare event particle in the absence of a rare event particle is less than 33%.

89. The method according to claim 84, where the reliability of the correlation of spatial image data to the number of rare event particles, defined as the probability of identifying a rare event particle in the presence of a rare event particle is better than 33%.

90. The method according to claim 84, where the steps are repeated a number of times until a predetermined volume of sample has been analysed.

91. The method according to claim 90, where the predetermined volume of sample is from 10 to 100 μl.

92. The method according to claim 90, where the predetermined volume of sample is more than 10 μl.

93. The method according to claim 84, where the steps are repeated until at least one rare event particle has been detected.

94. The method according to claim 84, where the steps are repeated until the absence of a rare event particle has been observed a pre-determined number of times or for a pre-determined sample volume.

95. The method according to claim 90, where the repetitions comprise serial repetitions in time.

96. The method according to claim 93, where the repetitions comprise serial repetitions in time.

97. The method according to claim 94, where the repetitions comprise serial repetitions in time.

98. The method according to claim 90, where the repetitions comprise parallel repetitions.

99. The method according to claim 93, where the repetitions comprise parallel repetitions.

100. The method according to claim 94, where the repetitions comprise parallel repetitions.

101. The method according to claim 84, where the steps are repeated at least 3 times.

102. The method according to claim 84, where the steps are repeated 20 to 100 times.

103. The method according to claim 84, where the detection of signals in step iii) is carried out for a period of time, being an exposure time.

104. The method according to claim 103, where the exposure time is less than 120 sec.

105. The method according to claim 103, where the particles move less than a distance corresponding to 150% of their diameter in a direction substantially parallel to the plane of the detection elements during the exposure time.

106. The method according to claim 103, where the particles move less than a distance causing the representation of the particles in the spatial image to move in the image corresponding to 150% of the diameter of the representation of the particle during the exposure time.

107. The method according to claim 105, where the percentage is less than 100%.

108. The method according to claim 106, where the percentage is less than 100%.

109. The method according to claim 84, where average particle diameter is less than 20 μm.

110. The method according to claim 84, where the precisely defined volume of the exposing domain in one dimension is substantially defined by walls.

111. The method according to claim 84, where the precisely defined volume of the exposing domain in one dimension is defined by walls being substantially parallel to the plane of the detection elements and the area viewed by the detection elements.

112. The method according to claim 84, where the precisely defined volume of the exposing domain is defined by walls being substantially parallel to the plane of the detection elements and a mask defining an area to be viewed by the detection elements.

113. The method according to claim 84, where the precisely defined volume of sample in the exposing domain is from 0.1 to 100 μl.

114. The method according to claim 84, where the rare event particle comprises a cell.

115. The method according to claim 84, where the precisely defined volume of sample in the exposing domain is from 0.1 to 5 μl.

116. The method according to claim 84, where the rare event particle comprises a bacterium.

117. The method according to claim 84, where the arrangement of a precisely defined volume of sample comprises replacement of a volume of sample in the exposing domain.

118. The method according to claim 84, where the arrangement of a precisely defined volume of sample comprises movement of the detection elements or the mask defining an area to be viewed relatively to the sample device.

119. The method according to claim 84, further comprising selective labelling of the rare event particle(s) before arranging it in the sample compartment.

120. The method according to claim 119, where the selective labelling comprises staining of the rare event particles.

121. The method according to claim 120, where the staining comprises staining of the nucleus of the rare event particles.

122. The method according to claim 119, further comprising selective staining of particles in the sample being non-rare.

123. The method according to claim 119, comprising an antibody based stain.

124. The method according to claim 122, wherein the non-rare particles comprise maternal blood cells and the rare particles comprise foetal blood cells, or the non-rare particles comprise normal mammal tissue cells and the non-rare cells comprise cancer cells or micrometastases, or the non-rare particles comprise blood cells and the rare particles comprise bacteria, fungal cells or spores or virus or plasmodium.

125. The method according to claim 84, wherein the signal which is detected by detection device is a signal which is substantially caused by attenuation of electromagnetic signal, and/or by emission of electromagnetic irradiation by photoluminescence, the attenuation and/or the photoluminescence being associated to one or more molecules which is/are a part of the particle.

126. The method according to claim 125, wherein the particle is a somatic cell or bacterium, and wherein the molecules are DNA and/or proteins.

127. The method according to claim 84, wherein the signal which is detected by detection device substantially originates from one or several types of molecules of types which bind to, are retained within, or interact with, the particles, such molecules being added to the sample before or during exposure of electromagnetic signals, the molecules being molecules giving rise to one or several of the following phenomena: attenuation of electromagnetic radiation, photoluminescence when illuminated with electromagnetic radiation, scatter of electromagnetic radiation, or raman scatter.

128. The method according to claim 84, wherein one or more reaction components initially loaded in a compartment or flow channel part of the flow system of the device is one or more nucleic acid dyes and/or one or more potentiometric membrane dyes.

129. The method according to claim 128, wherein a nucleic acid dye or nucleic acid dyes is/are added in an amount of 0.3-30 μg per ml of the sample.

130. The method according to claim 128, wherein one or more nucleic acid dyes is/are selected from the group consisting of: phenanthridines, acridine dyes, cyanine dyes, indoles and imidazoles.

131. The method according to claim 128, wherein the nucleic acid dye added is propidium iodide.

132. The method according to claim 128, wherein any reaction component added has the effect of aiding in the binding of one or more dyes to a particle.

133. The method according to claim 128, wherein any reaction component added has the effect of physically or chemically stabilising a particle.

134. The method according to claim 132, wherein such reaction component is citric acid or a salt of citric acid.

135. The method according to claim 84, where the rare event particles are identified using knowledge about at least one morphology criterion for the rare event particle.

136. The method according to claim 135, where non-rare particles are distinguished from rare event particles using at least one distinguishing morphological criterion.

137. The method according to claim 84, combining selective labelling and at least one morphology criterion to distinguish rare event particles from non-rare particles.

138. The method according to claim 84, where the sample comprises blood, leukocyte-depleted blood or blood products, donor blood, a biopsy, urine, maternal blood, foetal blood.

139. The method according to claim 84, where the rare event particles comprise abnormal cells, cancer cells, micrometastasis, parasites, ova from parasites, blood cells, leucocytes, erythrocytes, blood plates, virus, fungus, fetal cells, foetal blood cells, proteinaceous casts.

140. The method according to claim 84, where the method further includes particle retaining means for the substantially reproducible pre concentration of the rare particle being assessed.

141. The method according to claim 140, where a particle being retained by the particle retaining means is released into substantially less volume than initially introduced to the particle retaining means before analysis.

142. The method according to claim 141, where the particle is assessed while still being retained on or in the particle retaining means.

143. The method according to claim 84, where the array of detection elements comprises a charge coupled device (CCD) or an array of light sensitive diodes.

144. The method according to claim 84, where the detection of electromagnetic signals comprises one frame grabbing action.

145. The method according to claim 84, where the detection of electromagnetic signals comprises at least two frame grabbing actions.

146. The method according to claim 145, comprising averaging of at least two grabbed frames.

147. The method according to claim 84, further comprising a filtration retaining the rare event particles of the liquid sample prior to arranging the sample in the sample compartment.

148. The method according to claim 84, where the ratio of a linear dimension of the image on the array of detection elements to the original linear dimension in the exposing domain is in the range from 10:1 to 1:10.

149. The method according to claim 148, where the ratio of a linear dimension of the image on the array of detection elements to the original linear dimension in the exposing domain is in the range from 1.5:1 to 1:2.

150. The method according to claim 84, wherein the number of detection elements, onto which the image of one rare event particle is exposed is in the range from 1 to 16.

151. A method for collection of a rare event particle comprising:
i) arranging a volume of a liquid sample of at least 0.1 μl in the exposing domain of a sample compartment,
ii) detecting the absence or presence of a rare event particle,
iii) in case of presence of at least one rare event particle, flowing the volume of sample to an outlet, obtaining a sample comprising at least one rare event particle,
iv) repeating steps ii) to iii) until at least a predetermined number of rare event particles is obtained or until a predetermined volume of a liquid sample has been analysed in the exposing domain.

152. A method for isolation of a rare event particle comprising:
i) arranging a volume of a liquid sample in the exposing domain of a sample compartment,
ii) detecting the absence or presence of a rare event particle,
iii) in case of presence of a rare event particle, flowing the volume of sample to an outlet, obtaining a sample comprising a rare event particle,
iv) diluting the sample containing collected rare event particles and arranging a volume of the diluted sample in the exposing domain of a sample compartment,
v) repeating steps ii) to iv) until the rare event particle(s) is/are essentially the only particle(s) in a volume, obtaining a sample comprising essentially only rare event particle(s).

153. The method according to claim 152, where the repetition of steps ii) to iv) are carried out in the sample compartment of step i) (serial operation).

154. The method according to claim 152, where the repetition of steps ii) to iv) are carried out in a different, often identical, sample compartment (parallel operation).

155. The method according to claim 151, where the detection of absence or presence of a rare event particle is performed by:
i) in a sample device arranging a precisely defined volume of at least 0.1 μl of a liquid sample in an exposing domain of a sample compartment, allowing electromagnetic radiation from the rare event particle(s) in the exposing domain to pass to the exterior,
ii) arranging the sample device in relation to a detection device so that signals from the exposing domain can pass to an array of detection elements in the detection device,
iii) detecting electromagnetic signals from the first volume of liquid sample in the exposing domain by forming a spatial image of the rare event particle(s) on the array of detection elements,
iv) repeating steps i) and iii) at least once for new volumes of the same liquid sample, and
v) correlating the spatial image to the number of rare event particle(s) in the volume of liquid sample in the exposing domain.

156. The method according to claims 152, where the detection of absence or presence of a rare event particle is performed by:
i) in a sample device arranging a precisely defined volume of at least 0.1 μl of a liquid sample in an exposing domain of a sample compartment, allowing electromagnetic radiation from the rare event particle(s) in the exposing domain to pass to the exterior,
ii) arranging the sample device in relation to a detection device so that signals from the exposing domain can pass to an array of detection elements in the detection device,
iii) detecting electromagnetic signals from the first volume of liquid sample in the exposing domain by forming a spatial image of the rare event particle(s) on the array of detection elements,
iv) repeating steps i) and iii) at least once for new volumes of the same liquid sample, and
v) correlating the spatial image to the number of rare event particle(s) in the volume of liquid sample in the exposing domain.

157. The method according to claim 152, where the exposure time during the initial steps of collection or isolation are shorter than during the later steps of collection or isolation.

158. The method according to claim 152, further comprising filtration of the sample comprising the isolated rare event particle and diluted with carrier liquid, to reduce the volume of sample in which the rare event particle is present or to retain the rare event particle or a filter.

159. A system for collection of rare event particle(s) comprising:
i) a sample compartment comprising an exposing domain, from which electromagnetic radiation from a precisely defined volume of sample of more than 0.1 μl can pass to the exterior,
ii) a flow system comprising an inlet and an outlet, at least one of which comprises a stop valve,
iii) pumping means to pump liquid sample into and through the sample compartment,
iv) the flow system further comprising on the outlet side at least a waste outlet and a rare event particle outlet, as well as valve means to direct the sample to either of these outlets.

160. A system for isolation of a rare event particle comprising:
i) a sample compartment comprising an exposing domain, from which electromagnetic radiation from a precisely defined volume of sample can pass to the exterior,
ii) a flow system comprising an inlet and an outlet, at least one of which comprises a stop valve,
iii) pumping means to pump liquid sample or carrier liquid into and through the sample compartment,
iv) the flow system further comprising on the inlet side, at least a sample tube and a carrier liquid tube and valve means to connect the inlet to either of the tubes,
v) the flow system further comprising on the outlet side at least a waste tube and a rare event particle tube, as well as valve means to direct the sample to either of these tubes.

161. The system according to claim 159, further comprising tube means to connect the rare event particle tube on the outlet side to the sample inlet.

162. The system according to claim 160, further comprising tube means to connect the rare event particle tube on the outlet side to the sample inlet.

163. The system according to claim 159, wherein the precisely defined volume of sample in the exposing domain comprises from 0.1 to 1000 μl.

164. The system according to claim 160, wherein the precisely defined volume of sample in the exposing domain comprises from 0.1 to 1000 μl.

165. The system according to claim 159, wherein the precisely defined volume of the exposing domain in one dimension is defined by walls.

166. The system according to claim 160, wherein the precisely defined volume of the exposing domain in one dimension is defined by walls.

167. The system according to claim 159, wherein the precisely defined volume of the exposing domain is defined by walls being substantially parallel to the plane of the detection elements and the area viewed by the detection elements.

168. The system according to claim 160, wherein the precisely defined volume of the exposing domain is defined by walls being substantially parallel to the plane of the detection elements and the area viewed by the detection elements.

169. The system according to claim 159, wherein the precisely defined volume of the exposing domain is defined by walls being substantially parallel to the plane of the detection elements and a mask defining an area to be viewed by the detection elements.

170. The system according to claim 160, wherein the precisely defined volume of the exposing domain is defined by walls being substantially parallel to the plane of the detection elements and a mask defining an area to be viewed by the detection elements.

171. The system according to claim 159, further comprising detection means comprising an array of detection elements on which a spatial image of the rare event particle(s) in the exposing domain can be formed, as well as a data processor to process the detected images.

172. The system according to claim 160, further comprising detection means comprising an array of detection elements on which a spatial image of the rare event particle(s) in the exposing domain can be formed, as well as a data processor to process the detected images.

173. The system according to claim 171, comprising means to detect signals for a period of time, being an exposure time.

174. The system according to claim 172, comprising means to detect signals for a period of time, being an exposure time.

175. The system according to claim 173, wherein the exposure time is less than 120 sec.

176. The system according to claim 174, wherein the exposure time is less than 120 sec.

177. The system according to claim 159, wherein the array of detection elements comprise a charge coupled device (CCD) or an array of light sensitive diodes.

178. The system according to claim 160, wherein the array of detection elements comprise a charge coupled device (CCD) or an array of light sensitive diodes.

179. The system according to claim 159, wherein the detection of electromagnetic signals comprises one frame grabbing action.

180. The system according to claim 160, wherein the detection of electromagnetic signals comprises one frame grabbing action.

181. The system according to claim 159, wherein the detection of electromagnetic signals comprise at least two frame grabbing actions.

182. The system according to claim 160, wherein the detection of electromagnetic signals comprise at least two frame grabbing actions.

183. The system according to claim 181, comprising averaging of at least two grabbed frames.

184. The system according to claim 182, comprising averaging of at least two grabbed frames.

185. The system according to claim 159, further comprising means to filter a liquid sample comprising one rare event particle diluted with carrier liquid, while retaining the rare event particle.

186. The system according to claim 160, further comprising means to filter a liquid sample comprising one rare event particle diluted with carrier liquid, while retaining the rare event particle.

187. The system according to claim 159, further comprising at least one source of illumination to illuminate the sample in the exposing domain.

188. The system according to claim 160, further comprising at least one source of illumination to illuminate the sample in the exposing domain.

189. The system according to claim 187, wherein the source of illumination comprises light emitting diodes (LED), lasers, laser diodes, thermal light sources, gas discharge lamp, or stroboscopic light.

190. The system according to claim 188, wherein the source of illumination comprises light emitting diodes (LED), lasers, laser diodes, thermal light sources, gas discharge lamp, or stroboscopic light.
